(19) United States
(12) Patent Application Publication
(10) Pub. No.: US 2010/0311186 A1
(43) Pub. Date: Dec. 9, 2010

(54) DEVICES AND METHODS FOR PERFORMING RECEPTOR BINDING ASSAYS USING MAGNETIC PARTICLES

(75) Inventors: David M. Gregory, San Diego, CA (US); Joseph Michael Anderberg, Encinitas, CA (US)

Correspondence Address: WILSON, SONSINI, GOODRICH & ROSATI, 650 PAGE MILL ROAD, PALO ALTO, CA, 94304-1050, US

(73) Assignee: BIOSITE INCORPORATED, San Diego, CA (US)

(21) Appl. No.: 12/305,943

(22) PCT Filed: Jul. 27, 2007
(86) PCT No.: PCT/US2007/016909

§ 371 Date: Aug. 18, 2010

Related U.S. Application Data
(60) Provisional application No. 60/834,073, filed on Jul. 28, 2006.

(51) Int. Cl. G01N 33/53; G01N 21/00

(52) U.S. Cl. 436/501; 422/68.1; 422/82.05

(57) ABSTRACT

The present invention provides methods, devices, and systems for performing receptor binding assays. In particular, magnetically responsive particles configured to form a complex with a labeled conjugate corresponding to one or more analytes of interest can be moved within an assay device to one or more discrete detection regions through the application of one or more magnetic fields. By positioning the detection region such that the direction of this movement is, for at least a portion of the movement, counter to the direction of fluid flow within the device, detection of assay signals can be performed without the need for separate wash steps. Moreover, contamination of the signals resulting from labeled conjugate being carried in the direction of fluid flow substantially reduced.

### FIELD OF THE INVENTION

The present invention relates to systems and methods for conducting assays, including qualitative, semi-quantitative and quantitative determinations of one or more analytes wherein the analytes bind to magnetic particles and labeled conjugates comprising receptors corresponding to the analyte, detected by applying a magnetic field to bring the magnetic particles in proximity to a detector to generate a signal from the labeled conjugate.

### BACKGROUND OF THE INVENTION

The following discussion of the background of the invention is merely provided to aid the reader in understanding the invention and is not admitted to describe or constitute prior art to the present invention.

The term “receptor binding assay” refers to methods for generating a detectable signal indicative of the presence or amount of an analyte of interest based upon the ability of the analyte to bind with specificity to a particular binding partner (referred to as a “receptor” for the analyte). A common type of receptor binding assay is the immunoassay, in which antibodies that bind the analyte of interest are used to provide the analyte receptor, and the detectable signal is related to formation of an analyte/antibody complex. Numerous competitive, noncompetitive, and sandwich receptor binding assay methods are well known in the art. In addition to the use of antibodies as receptors for an analyte, the use of other binding partners, including nucleic acids, aptamers, and peptides other than those comprising an immunoglobulin motif. This list is not meant to be limiting.

Numerous methods, devices, and instruments are also well known in the art for practicing such receptor binding assays. See, e.g., U.S. Pat. Nos. 6,143,576; 6,113,855; 6,019,944; 6,007,690, 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety, including all tables, figures and claims. Suitable devices and instruments are also described in Chapter 41, entitled “Near Patient Tests: Triage® Cardiac System,” in The Immunoassay Handbook, 2nd ed., David Wild, ed., Nature Publishing Group, 2001, which is hereby incorporated by reference in its entirety, and in commonly owned U.S. Pat. No. 6,905,882, which is hereby incorporated by reference in its entirety, including all tables, figures and claims. One skilled in the art also recognizes that robotic instrumentation, including but not limited to Beckman ACCESS®, Abbott AXSYM®, Roche ELECSYS®, Dade Behring STRATUS® systems, are among the commercially available analyzers that are capable of performing receptor binding assays. Additionally, certain methods and devices, such as biosensors and optical immunoassays, can be employed to determine the presence or amount of analytes. See, e.g., U.S. Pat. Nos. 5,631,171 and 5,955,377, each of which is hereby incorporated by reference in its entirety, including all tables, figures and claims.

In such assay devices, flow of a sample fluid and other reagents along a desired flow path can be driven passively (e.g., by capillary, hydrostatic, or other forces that do not require further manipulation of the device once sample is applied), actively (e.g., by application of force generated via mechanical pumps, electroosmotic pumps, centrifugal force, increased air pressure, etc.), or by a combination of active and passive driving forces. Additional elements, such as filters to separate plasma or serum from blood, mixing chambers, etc., can be included as required by a particular application.

Binding of the analyte to its receptor can be detected directly or indirectly, and often employing the use of detectable labels. Such labels can be conjugated to receptors or to competitive receptor ligands, depending upon the type of assay being performed. The term “direct label” as used herein refers to a signal development element from which a signal can be generated without the addition of a further binding molecule that specifically binds one or more components of the analyte/receptor complex being detected. Examples of such direct labels include enzyme labels, fluorescent labels, electrochemical labels, metal chelates, colloidal metal labels, and biosensors relying on optical detection such as surface plasmon resonance and ellipsometry. Conversely, the term “indirect label” refers to a signal development element that binds, not to the analyte, but to a molecule that is itself bound to the analyte. A labeled secondary antibody, for example a detectably labeled goat anti-mouse IgG that binds to a mouse antibody directed to the analyte of interest, is an example of an indirect label.

In performing receptor binding assays, receptors (e.g., antibodies) are often immobilized on solid-phase matrices for use as affinity supports or to simplify sample analysis. The term “solid phase” as used herein refers to a wide variety of materials including solids, semi-solids, gels, films, membranes, meshes, felts, composites, particles, papers and the like typically used by those of skill in the art to sequester molecules. The solid phase can be non-porous or porous. Suitable solid phases include those developed and/or used as solid phases in solid phase binding assays. See, e.g., chapter 9 of Immunoassay, E. P. Dianiandis and T. K. Christopoulos eds., Academic Press, New York, 1996; Leon et al., Bioorg. Med. Chem. Lett. 8, 2997 (1998); Kessler et al., Agnew. Chem. Int. Ed. 40, 165 (2001); Smith et al., J. Comb. Med. 1, 326 (1999); Orain et al., Tetrahedron Lett. 42, 515 (2001); Papanikos et al., J. Am. Chem. Soc. 123, 2176 (2001); Gottschling et al., Bioorg. Med. Chem. Lett. 11, 2997 (2001), each of which is hereby incorporated by reference in its entirety. Such solid phase matrices can be modified to provide linkage sites, for example by bromoacetylation, silation, addition of amino groups using nitric acid, and attachment of intermediary proteins, dendrimers and/or star polymers. This list is not meant to be limiting, and any method known to those of skill in the art can be employed.

Of particular interest to the present invention are solid-phase matrices that respond to magnetic fields. When a magnetically responsive material is placed under the influence of a magnetic field, the material will tend to move toward or away from the region where the magnetic field is the strongest. For example, paramagnetic and ferromagnetic materials move in the direction of increasing strength of the magnetic field, while diamagnetic materials, such as polystyrene, move in the direction of decreasing strength of the magnetic field. Superparamagnetism occurs when a normally ferromagnetic material is composed of very small crystallites (1-10 nm), in which thermal energy at relatively low temperatures is sufficient to change the direction of magnetization of the entire crystallite. The resulting fluctuations in the direction of magnetization cause the magnetic field to average to zero. Thus, the material behaves in a manner similar to paramagnetism, except that instead of each individual atom being independently influenced by an external magnetic field, the magnetic moment of the entire crystallite tends to align with the magnetic field. Because super-paramagnetic materials have no “memory,” but still have relatively high magnetic susceptibilities, these materials are favored for use as magnetic responsive materials. Magnetically responsive materials have been used as solid phases to facilitate washing to separate bound and unbound labeled reagent, to assist in movement of reagents through a device, and to sequester the material bound to the solid phase to a specific location for detection of an assay signal. See, e.g., International Publications WO87/07386; and WO2004/035217; U.S. Pat. Nos. 4,452,773; 5,238,815; 5,445,970; 5,498,815; and 5,279,936; Choi et al., Biomedical Microdevices 3: 191-200, 2001; Brunet et al., Micromanipulating Magnetic Particles in Microfluidic Systems; and Furlani and Ng, Phys. Rev. E 73: 061919 (2006), each of which is hereby incorporated by reference in its entirety, including all tables, figures and claims. For example, Hayes et al. describe an immunoassay wherein primary antibody-linked paramagnetic particles are formed into a packed bed within a microchannel to create a high surface-area-to-volume ratio to increase the interaction of flowed-through samples and reagents with the immobilized particles. Anal. Chem. 73, 5896 (2001), which is hereby incorporated by reference in its entirety.

Consequently, there is a need for methods, devices and assays that allow for the improved detection of analytes. The disclosure provides these and additional benefits.

### SUMMARY OF THE INVENTION

In a first aspect, the invention relates to methods for performing one or more assays for one or more analytes in a fluid sample. In various embodiments described in detail hereinafter, these methods comprise the following steps:
(a) Introducing a fluid sample into an assay device comprising a sample addition region to receive the fluid sample, a second device region discrete from, and in fluid communication with, the sample addition region, and an analyte detection region discrete from, and in fluid communication with, both the sample addition region and the second device region. The second device region comprises a population of labeled conjugates corresponding to one or more analytes of interest. The assay device is configured to provide, upon application of the fluid sample to the sample addition region, fluid flow from the sample addition region to the second device region, and fluid flow from said sample addition region to said analyte detection region. In this manner, the second device region and the analyte detection region become fluidly connected. Furthermore, at least a portion of the fluid sample contacts the labeled conjugates in the second device region.
(b) Contacting the labeled conjugates with a population of magnetically responsive particles in the presence of at least a portion of the fluid sample, thereby forming a reaction mixture in said second device region. These magnetically responsive particles are configured to form a complex with the labeled conjugates in an amount related to the presence or amount of the analyte of interest in the reaction mixture.
(c) Following the contacting step, the magnetically responsive particles, now bound to labeled conjugates, can be separated from the reaction mixture by applying a magnetic field to the assay device. The magnetic field is configured to induce movement of the magnetically responsive particles on a path from the second device region to the analyte detection region. The direction of this movement is configured to be different from, and in certain embodiments counter to, to the direction of the fluid flow in part (a) of the method from the sample addition region to the second device region for at least a portion of this path. A signal is detected from labeled conjugates in the analyte detection region.

In another aspect, the invention relates to devices for performing the methods described herein. In various embodiments described in detail hereinafter, these devices comprise the following elements:
(a) a sample addition region to receive a fluid sample to be assayed for the presence or amount of one or more analytes of interest;
(b) a second device region discrete from, and in fluid communication with, the sample addition region, where the second device region comprises a population of labeled conjugates corresponding to at least one analyte of interest; and
(c) an analyte detection region discrete from, and in fluid communication with, both the sample addition region and the second device region, where the analyte detection region is positioned such that a movement path from the second device region to the analyte detection region is counter to the direction of fluid flow for at least part of the movement path; and
(d) magnetically responsive particles disposed within the device, where the particles comprise receptors immobilized thereon so as to be configured to form a complex with the labeled conjugates during performance of said assay.

In a related aspect, the invention relates to assay systems for performing the methods described herein. In various embodiments described in detail hereinafter, these systems comprise the following elements:
(a) an assay device as described above, where the labeled conjugates comprise a label moiety that generates a detectable optical signal upon illumination with electromagnetic energy having a wavelength that is absorbed by said label moiety, and where the device comprises a window or opening permitting illumination of the analyte detection region by an external source of electromagnetic energy; and
(b) an assay instrument comprising:
   (i) a receptacle for receiving the assay device;
   (ii) a magnetic field source that generates a magnetic field having an intensity sufficient to induce movement of the magnetically responsive particles on a path from the second device region to the analyte detection region during performance of an assay;
   (iii) a source of electromagnetic energy configured to illuminate the analyte detection region during performance of an assay to generate a detectable optical signal from the labeled conjugates in the analyte detection region; and
   (iv) a detector configured to receive the detectable optical signal and generate an electronic signal in response thereto.

Other embodiments of the invention will be apparent from the following detailed description, exemplary embodiments, and claims.

### DESCRIPTION OF THE FIGURES

FIG. 1 is a partially schematic, top perspective view of an assay device described in U.S. Pat. No. 5,458,852.

FIG. 2 is a schematic view of an assay device showing the spatial arrangement of device regions in one embodiment of the present invention.

FIG. 3 is a schematic view of an assay device showing the spatial arrangement of device regions in alternative embodiments of the present invention.

FIG. 4 is a schematic view of an assay device showing the spatial arrangement of device regions in an embodiment of the device for performing multiplexed assays.

FIG. 5 is a diagram illustrating a representative functional architecture of a fluorometer according to one embodiment of the present invention.

FIG. 6 is a diagram illustrating a representative functional architecture of the assay mechanism according to one embodiment of the present invention.

FIG. 7 is a graph showing BNP assay response data from an experiment of one embodiment of the present invention using stationary magnet field gradients to move the magnetically responsive particles.

FIG. 8 is a machine drawing of one embodiment of the present invention of a fixture used to hold two permanent magnets located in the square shaped openings and held in place with 4-40 set screws, two per magnet with a 0.125 inch wide slot allowing an embodiment of an assay device invention to pass between the magnets.

FIG. 9 is a graph showing BNP assay response data from the experiments of one embodiment of the present invention using a magnetic trap created by two magnets that move with respect to one embodiment of the assay device invention.

FIG. 10 is a graph showing BNP assay response data verses incubation time for four different BNP concentrations of one embodiment of the present invention, from experiments performed using a magnetic trap created by two magnets that move with respect to one embodiment of the assay device invention.

FIG. 11 is a schematic drawing of a test sequence performed using a device and instrument of the present invention.

FIG. 12 shows the magnet trap used in Examples 4 and 5. The dimensions are in mm. Labels on the left figure correspond to: A. Magnets; B. Aluminum spacers; and C. Iron Bridge.

FIG. 13 is a graph showing BNP assay response data from the experiments of one embodiment of the present invention using a magnetic trap created by two magnets and an iron bridge that moved with respect to one embodiment of the assay device invention.

FIG. 14 is a plot showing BNP assay response data from the experiments of one embodiment of the present invention using a magnetic trap created by two magnets and an iron bridge that moved with respect to one embodiment of the assay device invention. Circle represent data acquired with a BNP concentration of 223 pg/ml; squares represent data with BNP concentration<5 pg/ml.

FIG. 15 is a graph showing BNP assay response data from the experiments of one embodiment of the present invention using a magnetic trap created by two magnets and an iron bridge that moved with respect to one embodiment of the assay device invention. The data resulted from magnetic beads that had been dried and then brought back up into solution.

### DETAILED DESCRIPTION OF THE EMBODIMENTS

Disclosed herein are methods, devices, and instruments for performing receptor binding assays. In particular, magnetically responsive particles configured to form a complex with a labeled conjugate corresponding to an analyte of interest are moved from a reaction mixture comprising a sample fluid and the labeled conjugate to one or more discrete detection regions through the application of one or more magnetic fields. By positioning the detection region such that the direction of this movement is, for at least a portion of the movement, different from the direction in which fluid flow occurs as fluid fills the device, detection of an assay signal can be performed without the need for a separate wash step to separate bound and free label.

The direction of movement of the magnetically responsive particles to the detection region(s) can be counter to the direction of fluid flow as fluid enters and fills the portion of the device comprising the labeled conjugate (e.g., the “second device region”). As discussed hereinafter, this is not to imply that fluid is necessarily flowing at the same time that the magnetically responsive particles are moved to the detection region(s). Rather, the device is preferably configured such that the force providing flow of unbound labeled conjugate from the second device region (that is, the portion of the device initially comprising the labeled conjugate) to the detection region(s) is the diffusion rate of the labeled conjugate in the fluid filling the device. Said another way, the flow of fluid through the device does not “wash” the unbound labeled conjugate from the second device region towards the detection region(s). Movement of the magnetic particles from the reaction mixture to the detection region(s) is preferably fast enough that the magnetic particles arrive at the detection region faster than the diffusion rate of the labeled conjugate can contribute to a background signal, thereby reducing background signal derived from unbound labeled conjugate. In certain embodiments, movement of the magnetically responsive particles is performed when fluid flow has a velocity of zero, such as occurs subsequent to filling of the device with fluid.

Preferably, the devices of the present invention comprise at least one chamber that is “substantially flattened,” and most preferably “substantially elongated,” through which the magnetic particles are moved. Each of these terms is defined hereinafter. By providing a chamber having a long axis in the direction of fluid flow, the length of which is at least 10 times the length of one, and most preferably both, other chamber axes (in a 3-dimensional Cartesian coordinate system), the chamber configuration can minimize mixing caused by movement of the magnetic particles through the device, and thus minimize background signal moving against the direction of flow. While not wishing to be held to a particular theory, it is believed that this is because frictional drag between the fluid filling the chamber and the chamber walls can act to counterbalance backflow around the particles.

As described herein, contamination of the signal by a nonspecific signal at the analyte detection region resulting from labeled conjugate being carried in the direction of fluid flow is substantially reduced. This can reduce or eliminate the need for wash steps that are common to many methods that incorporate magnetically responsive solid-phase matrices. The methods, devices, and instruments described herein can meet the need in the art for rapid and sensitive receptor binding assays.

As used herein, “velocity” is a vector quantity whose magnitude is a body's speed and whose direction is the body's direction of motion. Static flow is when the average velocity of fluid within a device is zero. The direction and speed at which the magnetic field(s) direct label to be detected may optimally be selected to deliver label to a detection location faster than the background signal could arrive at the same location due to forces such as diffusion. For example, flow of material can be in the reverse direction of one dimension flow of a fluid, in a direction that is in a different direction of the fluid within a two dimensional plane, or in a direction out of the two dimensional plane of the fluid. Furthermore, flow of material can be at a different velocity, slower or faster, than fluid flow.

As used herein with regard to movement of material other than fluid (e.g., magnetically responsive particles) within a device, the term “counter to the direction of fluid flow” refers to movement (e.g., of magnetically responsive particles) along a path from a first location in the device to a second location in the device, where the path comprises movement in one or more directions, and where the positions of the first and second locations are configured such that, when fluid is introduced into the device at the sample addition region, fluid flows from the second location to the first location. As discussed above in regard to the static flow condition, this is not meant to imply that flow is counter to the direction that fluid is flowing at every instant. Instead, the term refers to the direction that fluid flow occurs or occurred when fluid filled the space between the second location and the first location.

To achieve minimal sample volumes, mesoscale assay devices can be used. The term “mesoscale” as applied to the assay devices of the present invention refer to devices in which fluid flow occurs through one or more chambers having one or more cross-sectional dimensions that are between 0.1 μm and 500 μm. This is not meant to imply that such a chamber is mesoscale in all dimensions. For example, a chamber can be elongate, in that the dimension from a first location to a second location is on the scale of millimeters, centimeters, or greater, while the height and/or width is mesoscale. Alternatively, a chamber can be dimensioned on the scale of millimeters, centimeters, or greater in both length and width, but mesoscale in height. In this discussion, length, width, and height are used for the sake of convenience, with each simply intended to refer to one axis of a three-dimensional coordinate system. By using elongate chambers, such that the dimension between that portion of the device initially comprising the labeled conjugate (e.g., the “second device region”) and that portion of the device comprising the detection region(s) is greater than mesoscale, a device can increase the distance across which unbound labeled conjugate must diffuse to create a background signal at the detection region(s). It is particularly preferred that at least one second device region and at least one analyte detection region are within mesoscale chambers, and most preferably at least one second device region and at least one analyte detection region are within a single, preferably substantially elongate, mesoscale chamber. A plurality of such elongate chambers may be fluidly connected to a single sample addition region as described hereinafter.

The term “chamber” as used in this context refers to an enclosed cavity having one or more openings for fluid ingress and/or egress. A chamber is distinguished from a fibrous or porous matrix, such as a filter or membrane, that imbibe, or “wick,” fluid into numerous internal voids. The mesoscale assay devices of the present invention are sometimes referred to as “capillary” devices, as such mesoscale dimensions in one or more chambers of the device can be used to provide fluid flow that is mediated in whole or in part by capillary force.

This is also not intended to imply that all chambers of a mesoscale assay device must have at least one mesoscale cross-sectional dimension. Thus, a chamber of mesoscale dimensions can be connected to one or more chambers of larger dimensions, for example to receive the initial sample prior to entry to a mesoscale chamber, and/or to receive sample outflow from a mesoscale chamber. Nor is it intended to imply that mesoscale assay devices cannot include, in addition to one or more chambers, one or more fibrous or porous matrices. For example, a fibrous or porous filter can be provided to remove particulate matter (e.g., cells such as erythrocytes from blood) prior to entry to a mesoscale chamber, and/or a fibrous or porous member can be provided to receive sample outflow from a mesoscale chamber.

In suitable embodiments, the second device region and/or the analyte detection region is/are within the same chamber, or separate chambers, having at least one dimension less than 500 μm, preferably less than 250 μm, and still more preferably less than 100 μm. In suitable embodiments, mesoscale chamber(s) are substantially flattened or substantially elongated. These terms as used herein refer to a chamber having an aspect ratio of at least 5, at least 10, at least 20, at least 50, or at least 100 or more. The “aspect ratio” of a three-dimensional shape is the ratio of its longest dimension to its shortest dimension. A chamber is “substantially flattened” if the ratio of the longest axis to the shortest axis (using a 3-dimensional Cartesian coordinate system) is at least 10, more preferably at least 20, and most preferably at least 50. A substantially flattened chamber is “substantially elongated” if the longest axis is on the same axis as that of the axis of fluid flow, and the ratio of the longest axis to each of the other two axes is at least 10, more preferably at least 20, and most preferably at least 50. The substantially elongate shape (which can be referred to as a “lane”) in particular can reduce background signal reaching the “upstream” portions of the device by lengthening the distance that such background signal must traverse to be detected. It is believed that this is because frictional drag between the fluid filling the chamber and the chamber walls can act to counterbalance backflow around the particles. This can act to constrain fluid motion during particle motion.

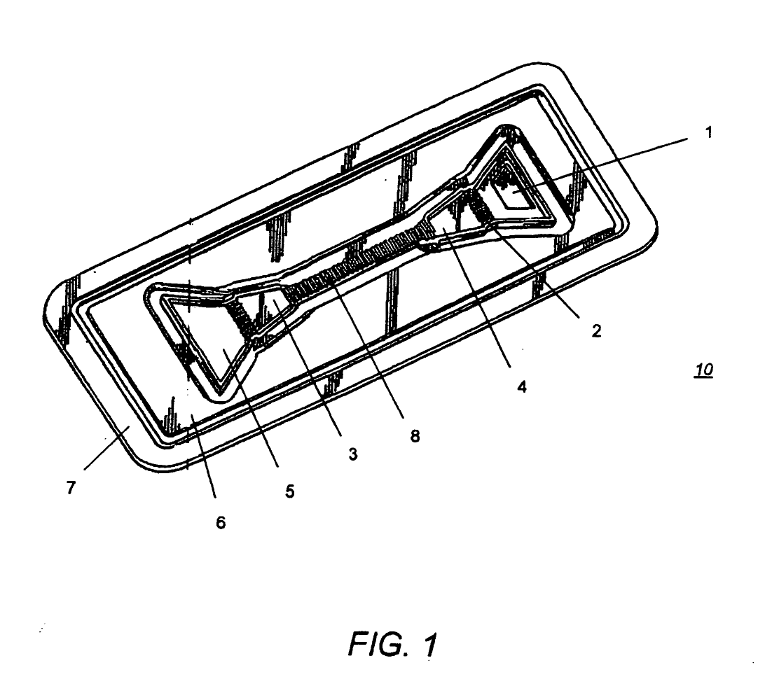

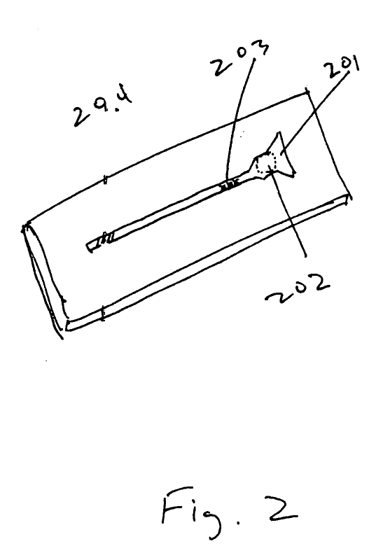

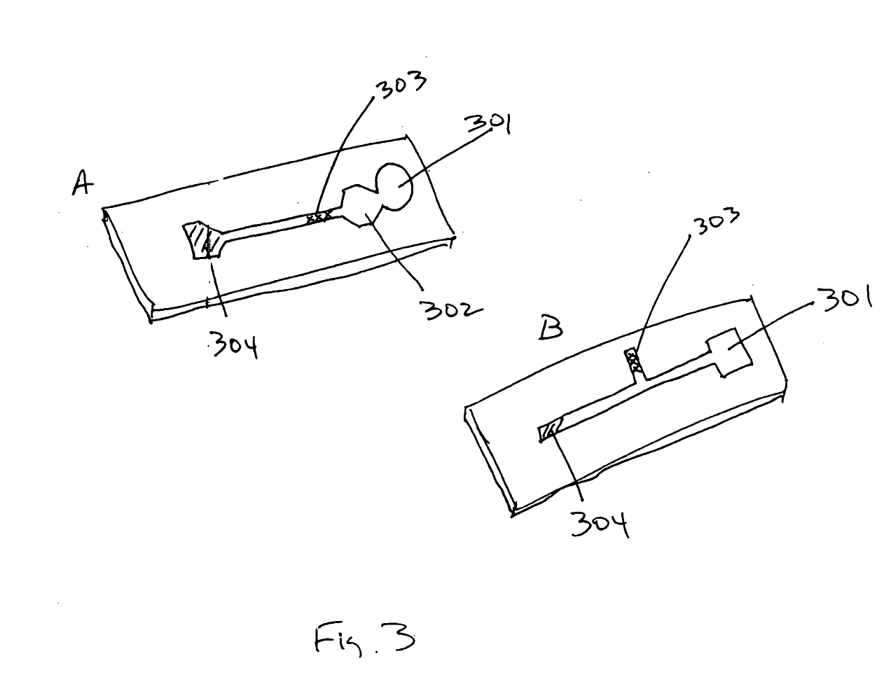

The present invention is directed to diagnostic testing devices, systems, and methods for determining the presence or amount of at least one target ligand (i.e., analyte). FIGS. 1 and 2 show an embodiment of an assay device 10 according to the invention. The device 10 can comprise various elements, including: a sample addition region 1, a sample reaction barrier 2, a second device region 3, an analyte detection region 4, and a used reagent reservoir 5. The devices can be comprised of capillary channels which are formed when a top member 6 is placed on the bottom member 7 a capillary distance apart and which move the reagents and sample throughout the device. The top and bottom members can be married, the various chambers sealed and the capillaries formed by a number of techniques, including but not limited to, gluing, welding by ultrasound, riveting and the like. The elements of the device can be used in various combinations with the analyte detection region 4 to achieve a variety of desired functions. As one skilled in the art will recognize these elements can be combined to perform one-step or multistep assays. The devices 10 can also be used in the formation of reaction mixtures for the assay process. An optional reagent chamber 17 can be incorporated into device 10 as depicted in FIG. 3.

Components of the device (i.e. a physical structure of the device whether or not a discrete piece from other parts of the device) can be prepared from copolymers, blends, laminates, metallized foils, metallized films or metals. Alternatively, device components can be prepared from copolymers, blends, laminates, metallized foils, metallized films or metals deposited one of the following materials: polyolefins, polyesters, styrene containing polymers, polycarbonate, acrylic polymers, chlorine containing polymers, acetal homopolymers and copolymers, cellulosics and their esters, cellulose nitrate, fluorine containing polymers, polyamides, polyimides, polymethylmethacrylates, sulfur containing polymers, polyurethanes, silicon containing polymers, glass, and ceramic materials.

Alternatively, components of the device are made with a plastic, elastomer, latex, silicon chip, or metal; the elastomer can comprise polyethylene, polypropylene, polystyrene, polyacrylates, silicon elastomers, or latex.

Alternatively, components of the device can be prepared from latex, polystyrene latex or hydrophobic polymers; the hydrophobic polymer can comprise polypropylene, polyethylene, or polyester.

Alternatively, components of the device can comprise TEFLON®, polystyrene, polyacrylate, or polycarbonate. Alternatively, device components are made from materials such as plastics which are capable of being milled or injection molded or from surfaces of glass, silicon, copper, silver and gold films. The materials which are capable of being milled or injection molded can comprise a polystyrene, a polycarbonate, or a polyacrylate.

The term “reaction mixture” as used herein refers to the mixture of fluid sample suspected of containing target analytes, and one or more reagents for determining the presence or amount of analytes in the sample. For example, the reaction mixture might comprise one or more ligand analogue conjugates or receptor conjugates corresponding to one or more analytes of interest, and/or the magnetically responsive particles comprising receptors corresponding to one or more analytes of interest. As used herein, the reaction mixture can comprise additional components, including for example buffering agents, HAMA inhibitors, detergents, salts (e.g., chloride and/or sulfate salts of calcium, magnesium, potassium, etc.), proteinaceous components (e.g., serum albumin, gelatin, milk proteins, etc.). This list is not meant to be limiting.

With regard to receptors and/or labeled conjugates, the phrase “corresponding to an analyte of interest” refers to a receptor and/or labeled conjugate used in the method to generate a signal indicative of the presence or amount of the analyte in the reaction mixture. Depending on the receptor binding assay format being performed, the labeled conjugate can comprise a detectable label conjugated to a receptor that binds the analyte of interest (e.g., an anti-analyte antibody), can be a detectable label conjugated to a molecule that competes with the analyte of interest for binding to a receptor (e.g., an analyte analogue), or can be a detectable label conjugated to a binding partner that binds to a receptor for the analyte of interest (e.g., a secondary antibody, such as a goat anti-mouse IgG that binds to a mouse anti-analyte antibody). This list is not meant to be limiting. Numerous sandwich, competitive, and homogeneous receptor binding assay formats are known to those of skill in the art.

### Detectable Labels

As disclosed above, biological assays utilize various methods for detection, and one of the most common methods for quantitation of results is to conjugate an enzyme, fluorophore or other detectable label to the molecules under study (e.g., one or more analyte analogues), which can be immobilized for detection by receptors that have affinity for the molecules. Alternatively, the receptors to the one or more analytes of interest (e.g., an antibody or binding fragment thereof made or selected using the analyte of interest) can be conjugated to an enzyme, fluorophore or other detectable label. Enzyme conjugates are among the most common conjugates used. Detectable labels can include molecules that are themselves detectable (e.g., fluorescent moieties, electrochemical labels, metal chelates, etc.) as well as molecules that can be indirectly detected by production of a detectable reaction product (e.g., enzymes such as horseradish peroxidase, alkaline phosphatase, etc.) or by a specific binding molecule which itself may be detectable (e.g., biotin, digoxigenin, maltose, oligohistidine, 2,4-dintrobenzene, phenylarsenate, ssDNA, dsDNA, etc.).

A variety of linkage chemistries have been described for the attachment of a detectable label to a particular molecule of interest, often for purposes developing binding assay (e.g., immunoassay) reagents. Thus, molecules may be coupled via a selected linkage chemistry for solid-phase immobilization, preparation of antibody-detectable label conjugates and other labeled protein and nucleic acid reagents, etc. Such linkage chemistries often provide the molecule of interest with one or more functional groups that couple to amino acid side chains of peptides. Among other characteristics, these “linkage reagents” may be classified on the basis of the following:

1. Functional group(s) and chemical specificity;

2. length and composition of the cross-bridge;

3. whether the functional group(s) react chemically or photochemically; and

4. whether the resultant linkage is cleavable.

Reactive groups that can be targeted using linkage chemistries include primary amines, sulfhydryls, carbonyls, carbohydrates and carboxylic acids. In addition, many reactive groups can be coupled nonselectively using a cross-linker such as photoreactive phenyl azides.

Linkage chemistries may be provided with a variety of spacer arm (or “bridge”) lengths for spacing the molecule of interest from its conjugate partner. The most apparent attribute of the bridge is its ability to deal with steric considerations of the moieties to be linked. Because steric effects dictate the distance between potential reaction sites, different lengths of bridges may be considered for the interaction. Suitable linkers are well known in the art, and are commercially available from companies such as Pierce Biotechnology, Inc. (Rockford, Ill.).

Preferred detectable label conjugates are less than about 100 nm in size, more preferably less than about 70 nm in size, still more preferably less than about 40 nm in size, and most preferably less than about 20 nm in size. The term “about” as used in this context refers to +/−10% of a given value. Certain preferred detectable labels include fluorescent latex particles such as those described in U.S. Pat. Nos. 5,763,189; 6,238,931; and 6,251,687; and International Publication WO95/08772, each of which is hereby incorporated by reference in its entirety.

The presence or amount of one or more analytes is preferably determined using antibodies specific for each analyte and detecting specific binding. Any suitable immunoassay can be utilized, for example, competitive and noncompetitive immunoassays, sandwich immunoassays, and the like. Specific immunological binding of the antibody to the analyte can be detected directly or indirectly using labels.

Numerous methods and devices are well known to the skilled artisan for the detection and analysis of the analytes of the instant invention. Flow of sample along the flow path within a device can be driven passively (e.g., by capillary, hydrostatic, or other forces that do not require further manipulation of the device once sample is applied), actively (e.g., by application of force generated via mechanical pumps, electroosmotic pumps, centrifugal force, increased air pressure, etc.), or by a combination of active and passive driving forces. Various optional device elements, such as filters to separate plasma or serum from blood, mixing chambers, etc., can be included as required by the artisan. Exemplary devices are described in Chapter 41, entitled “Near Patient Tests: Triage® Cardiac System,” in The Immunoassay Handbook, 2^{nd} ed., David Wild, ed., Nature Publishing Group, 2001; and U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety, including all tables, figures and claims. These devices and methods can utilize labeled molecules in various sandwich, competitive, or non-competitive assay formats, to generate a signal that is related to the presence or amount of an analyte of interest.

### Selection of Receptors

A ligand-receptor pair refers to a ligand and receptor that are chemical moieties capable of recognizing and binding to each other. The ligand and receptor can be any moieties that are capable of recognizing and binding to each other to form a complex. Additionally, the ligand and receptor can interact via the binding of a third intermediary substance. Typically, the ligand and receptor constituting the ligand-receptor pair are binding molecules that undergo a specific noncovalent binding interaction with each other. The ligand and receptor can be naturally occurring or artificially produced, and optionally can be aggregated with other species.

Examples of ligands and/or receptors include, but are not limited to, agonists and antagonists for cell membrane receptors, toxins and venoms, viral epitopes, hormones such as steroids, hormone receptors, peptides, enzymes and other catalytic polypeptides, enzyme substrates, cofactors, drugs including small organic molecule drugs, opiates, opiate receptors, lectins, sugars, saccharides including polysaccharides, proteins, and antibodies including monoclonal antibodies and synthetic antibody fragments, cells, cell membranes and moieties therein including cell membrane receptors, and organelles. Examples of ligand-receptor pairs include lectin-carbohydrate; peptide-cell membrane receptor; protein A-antibody; hapten-antihapten; digoxigenin-anti-digoxigenin; enzyme-cofactor; enzyme-substrate; and antibody-antigen. As used herein, analytes can be ligands or can be associated with a ligand. Thus, where the analyte is an antigen, an antibody that binds to the antigen is a receptor.

The generation and selection of antibodies can be accomplished several ways. For example, one way is to purify polypeptides of interest or to synthesize the polypeptides of interest using, e.g., solid phase peptide synthesis methods well known in the art. See, e.g., Guide to Protein Purification, Murray P. Deutcher, ed., Meth. Enzymol. Vol 182 (1990); Solid Phase Peptide Synthesis, Greg B. Fields ed., Meth. Enzymol. Vol. 289 (1997); Kiso et al., Chem. Pharm. Bull. (Tokyo) 38, 1192 (1990); Mostafavi et al., Biomed. Pept. Proteins Nucleic Acids 1, 255 (1995); Fujiwara et al., Chem. Pharm. Bull. (Tokyo) 44, 1326 (1996). The selected polypeptides can then be injected, for example, into mice or rabbits, to generate polyclonal or monoclonal antibodies. One skilled in the art will recognize that many procedures are available for the production of antibodies, for example, as described in Antibodies, A Laboratory Manual, Ed Harlow and David Lane, Cold Spring Harbor Laboratory, Cold Spring Harbor, N.Y., 1988. One skilled in the art will also appreciate that binding fragments or Fab fragments which mimic antibodies can also be prepared from genetic information by various procedures (Antibody Engineering: A Practical Approach, Borrebaeck, C., ed., Oxford University Press, Oxford, 1995; J. Immunol. 149, 3914 (1992)).

In addition, numerous publications have reported the use of phage display technology to produce and screen libraries of polypeptides for binding to a selected target. See, e.g., Cwirla et al., Proc. Natl. Acad. Sci. USA 87, 6378 (1990); Devlin et al., Science 249, 404 (1990); Scott & Smith, Science 249, 386 (1990); and Ladner et al., U.S. Pat. No. 5,571,698. A basic concept of phage display methods is the establishment of a physical association between DNA encoding a polypeptide to be screened and the polypeptide. This physical association is provided by the phage particle, which displays a polypeptide as part of a capsid enclosing the phage genome which encodes the polypeptide. The establishment of a physical association between polypeptides and their genetic material allows simultaneous mass screening of very large numbers of phage bearing different polypeptides. Phage displaying a polypeptide with affinity to a target bind to the target and these phage are enriched by affinity screening to the target. The identity of polypeptides displayed from these phage can be determined from their respective genomes. Using these methods a polypeptide identified as having a binding affinity for a desired target can then be synthesized in bulk by conventional means. See, e.g., U.S. Pat. No. 6,057,098, which is hereby incorporated in its entirety, including all tables, figures, and claims.

The antibodies that are generated by these methods can then be selected by first screening for affinity and specificity with the purified polypeptide of interest and, if required, comparing the results to the affinity and specificity of the antibodies with polypeptides that are desired to be excluded from binding. The screening procedure can involve immobilization of the purified polypeptides in separate wells of microtiter plates. The solution containing a potential antibody or groups of antibodies is then placed into the respective microtiter wells and incubated for about 30 minutes to 2 hours. The microtiter wells are then washed and a labeled secondary antibody (for example, an anti-mouse antibody conjugated to alkaline phosphatase if the raised antibodies are mouse antibodies) is added to the wells and incubated for about 30 minutes and then washed. Substrate is added to the wells and a color reaction will appear where antibody to the immobilized polypeptide(s) are present.

The antibodies so identified can then be further analyzed for affinity and specificity in the assay design selected. In the development of immunoassays for a target protein, the purified target protein acts as a standard with which to judge the sensitivity and specificity of the immunoassay using the antibodies that have been selected. Because the binding affinity of various antibodies can differ; certain antibody pairs (e.g., in sandwich assays) may interfere with one another sterically, etc., assay performance of an antibody may be a more important measure than absolute affinity and specificity of an antibody.

Those skilled in the art will recognize that many approaches can be taken in producing antibodies or binding fragments and screening and selecting for affinity and specificity for the various polypeptides, but these approaches do not change the scope of the invention.

### Magnetically Responsive Particles

Small polymer microspheres (i.e. beads of low-μm to sub-μm diameters) such as the magnetically responsive particles discussed above have been used as solid phase matrices in surface-binding assays. Magnetic beads can: (1) increase effective ratios of reactive surface to volume and make it possible to carry out reactions in much smaller volumes; (2) move easily for analyte transport and delivery; (3) substantially reduce diffusion distances through the narrow fluid paths between neighboring beads; and (4) enable localization of the biomolecular interaction to a specific point in the analysis system.

Numerous publications have reported the use of magnetically responsive particles in capillary flows, as described in part in Watari et al., Anal. & Bioanal. Chem. 378, 1693 (2004); Verpoorte, E., Lab Chip 3, 60N (2003); and U.S. Pat. Nos. 6,953,676; 5,222,808; and 5,145,784, each of which is hereby incorporated in its entirety.

Magnetically responsive particles, beads, can be commercially available from sources such as Dynal, Inc. (Lace Success, N.Y.), Miltenyi Biotec, Inc. (Auburn, Calif.), Applied Biosystems (previously PerSeptive Biosystems, Inc., (Foster City, Calif.), Bayer Diagnostics (Medfield, Mass.), Bangs Laboratories (Carmel, Ind.), and BioQuest, Inc. (Atkinson, N.H.). The particles can be made of materials such as iron, iron oxide, iron nitride, iron carbide, nickel and cobalt, as well as mixtures and alloys thereof. Magnetically responsive latex beads are available from commercial sources such as Seradyne (Indianapolis, Ind.).

In some cases the magnetically responsive particles are made from polystyrene, but cellulose, agarose, silica, porous glass or silanized particles are also available. Some commercially available particles are made of flakes of magnetic oxides of different sizes and shapes, with a layer of chemical groups on the surface. The particles can also be made by mixing small grains of magnetic oxides with natural or synthetic polymer, followed by procedures to achieve appropriate particle size. Magnetically responsive particles have also been prepared adding magnetic oxides to a mixture of highly water-insoluble compounds and vinyl monomers. The aqueous dispersion of droplets containing magnetic oxides can then be made into magnetically responsive particles by polymerization of the monomer. When used in, for example, immunoassay, the chemical composition of the particle surface is critical because an inert surface that does not bind to biological elements other than the analyte receptor is highly desirable. The magnetically responsive particles of certain embodiments of the current invention can be paramagnetic or super-paramagnetic, i.e., they are magnetic in a magnetic field but are non-magnetic as soon as the magnetic field is removed. Identical size and form of the particles are preferable in order to perform identically in a suspension, with respect to sedimentation and kinetics of binding to other molecules. The magnetically responsive particles can have a maximum length that is a small fraction of the length or width of the chamber in which mixing takes place. The magnetically responsive particles typically can have a size in the range of about 0.1-100 μm in diameter, about 1-50 μm, about 0.3-10 μm, about 0.5-5 μm, or about 1-5 μm. The shape of the magnetically responsive particles will commonly be spherical, but particles of other configurations, irregular, rod-like, etc., can also be used. The term “about” as used in this context means±10% of a given measurement.

A common characteristic to all of these magnetically responsive particles is that specific binding molecules (receptors) can be attached to them. The most frequently used molecules are antibodies. The attachment of the receptors can be achieved through covalent and non-covalent binding between the particle surface and the antibody by various linkage chemistries as discussed above, such as where specific chemical groups on the particle surface bind to, for example, —NH₂ or —SH groups on the receptors.

In various embodiments, the population of magnetically responsive particles can be introduced into the assay device prior to, together with, or after addition of the sample fluid to the assay device. In certain embodiments, the population can be located within any portion of the assay device prior to application of said fluid sample, as they can be transported to the second device region by application of a magnetic field if necessary. In various embodiments, the population of magnetically responsive particles is disposed in or on the second device region such that their movement by application of a magnetic field is not required to form the reaction mixture. In still other embodiments, the population of magnetically responsive particles is transported to the second device region at least in part by fluid flow occurring upon addition of the sample fluid to the assay device. In various embodiments, the population of magnetically responsive particles can be emplaced in the device as a slurry or suspension with an appropriate buffer, which may optionally be dried, for example by lyophylization or other means, to form a diffusible coating on one or more surfaces of the device. A “diffusible coating” is one that is resuspended upon contact or incubation with a fluid sample that is introduced into the device.

The magnetically responsive particles bound with the analyte receptor or secondary antibody act as a solid phase matrix which the analyte of interest or the analyte-receptor conjugate complex can interact with. As mentioned previously, the magnetically responsive particles can be configured to form a complex with the labeled conjugates in an amount related to the presence or amount of the analyte of interest in the reaction mixture, referred to herein as the “reaction complex” or “magnetically responsive particles complex.” Numerous strategies for configuring such magnetically responsive particles can be employed, depending on the receptor binding assay format being performed. For example, in one configuration of a non-competitive receptor binding assay, analyte receptors bound to the magnetically responsive particles may form a sandwich complex with the analyte of interest and labeled analyte receptor conjugate. In another configuration, a secondary antibody bound to the magnetically responsive particles may form a sandwich complex with the analyte of interest and labeled analyte receptor conjugate. In one configuration of a competitive receptor binding assay, the analyte of interest may compete with an analogue conjugated to a detectable label for binding to a receptor bound to the magnetically responsive particles. This list is not meant to be limiting.

### Exemplary Elements of the Assay Devices

A device preferably comprises a sample addition region, a second device region discrete from, and in fluid communication with, the sample addition region; and an analyte detection region discrete from, and in fluid communication with, both the sample addition region and the second device region, where the analyte detection region is positioned such that a movement path from the second device region to the analyte detection region is counter to the direction of fluid flow for at least part of the movement path. In addition to these elements, other optional elements are discussed hereinafter. FIG. 1 shows a schematic drawing of a capillary assay device as described in U.S. Pat. No. 5,458,852, which is hereby incorporated in its entirety. Many of the features commonly finding use in capillary assay devices are described in this patent, and are discussed in general terms in the following sections.

The surface of any region within the devices described herein can be smooth (relative to the dried reagent deposit) or comprised of texture structures such as posts or grooves. Texture on a device surface can facilitate drying of a reagent or reagents (and can include magnetically responsive particles) during preparation of the device, and can facilitate movement of sample into the region. Texture on a device surface facilitates uniform placement of dried reagents on the surface as follows: A liquid reagent-containing fluid is placed in contact with the textured surface, and small reagent fluid menisci form adjacent each texture structure. Absent the presence of texture, the fluid would tend to form larger menisci at corners of the entire chamber, which when dried would produce a non-uniform layer of dried reagent. When texture structures are designed into the device, the presence of numerous small menisci leads to a more uniform layer of reagent that is dried throughout the chamber.

### Sample Addition Region

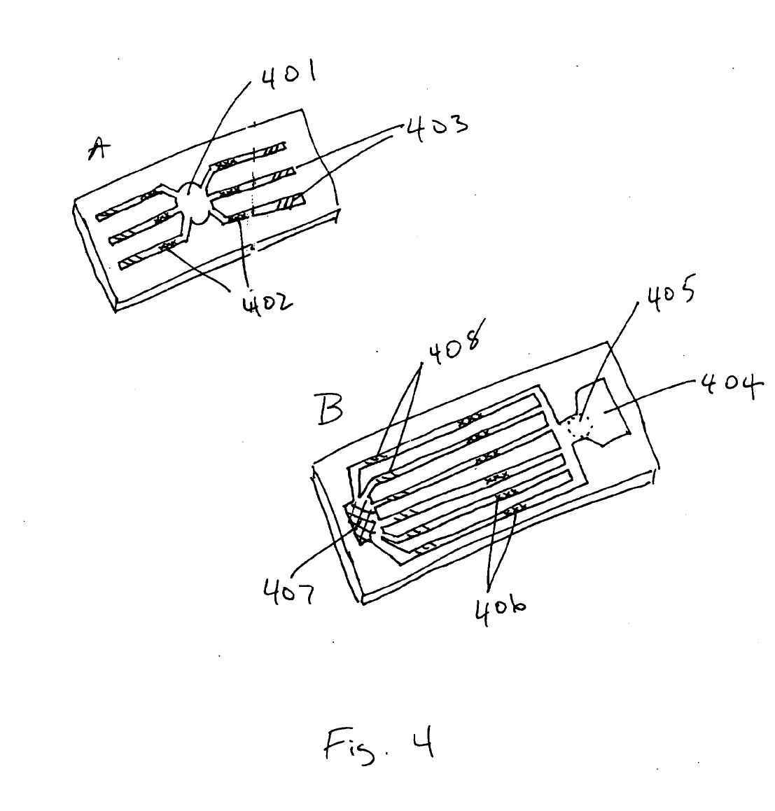

The sample addition region of capillary assay devices includes the area where sample is introduced to the device. Exemplary embodiments of the sample addition region are depicted as element 1 of FIG. 1, element 201 of FIG. 2, element 301 of FIG. 3, and elements 401 and 404 of FIG. 4. The sample addition region can be a port of various configurations, that is, round, oblong, square and the like or the region can be a trough in the device. In addition, a filter element can be placed in, on, or adjacent to the sample addition region to filter particulates from the sample or to filter blood cells from blood so that plasma can further travel through the device. Exemplary embodiments of such a filter element are depicted as element 202 of FIG. 2 and element 405 of FIG. 4. Suitable filters are well known in the art. See, e.g., U.S. Pat. No. 6,391,265, which is hereby incorporated by reference in its entirety. The sample addition region can comprise a vent (not illustrated) to facilitate escape of gas and liquid filling of the region. Alternatively, such a vent can be located in other regions of the device to facilitate filling of the capillary space(s).

The volume of the sample addition region can be at least the volume of the second device region or greater. The volume or capacity of the sample addition region can be 1 to 5 times the volume of the chamber(s) of the second device region and/or analyte detection region. In the exemplary devices described in Chapter 41, entitled “Near Patient Tests: Triage® Cardiac System, a volume or capacity of this sample addition region may be selected such that excess sample provides a wash to thoroughly remove any unbound reagents from the magnetically responsive particles and further bind target analytes to the magnetically responsive particles arising from the assay process. Because the present devices are configured to avoid the need for such a wash step, the capacity of this sample addition region may be substantially reduced to reduce the volume of sample required.

The sample addition region can also contain certain dried reagents which are used in the assay process. For example, a surfactant can be dried in this sample addition region which dissolves when sample is added. The surfactant in the sample would aid in the movement of the sample and reaction mixture through the device by lowering the surface tension of the liquid. The sample addition region can be placed in direct fluid contact with an optional sample-reaction barrier, with the second device region and/or with the analyte detection region.

### Sample Reaction Barrier

As depicted in FIG. 1, the sample reaction barrier 2 is an optional device element that can separate the excess sample in the sample addition region 1 from the portion of the sample forming a reaction mixture in distal regions of the device. Although the sample reaction barrier 2 can be optional in any embodiment, the sample reaction barrier 2 can provide the device with the capability of forming a precise reaction mixture volume.

The sample reaction barrier 2 can comprise a narrow capillary, generally ranging from about 0.01 mm to 0.2 mm and the surfaces of the capillary can be smooth or have a single groove or a series of grooves which are parallel or perpendicular to the flow of sample. In a suitable embodiment of the sample reaction barrier 2, grooves 12, parallel to the flow of sample, are incorporated onto one surface of the device a capillary distance, for example, 0.02 mm to 0.1 mm, from the other surface. The volume of sample which fills the sample reaction barrier 2 is preferably kept to a minimum, from about 0.01% to 10% downstream volume contained within the device so that the reagents present in distal regions of the device do not significantly diffuse back into the sample in the sample addition region 1. That is, the diffusion of the reaction mixture back into the excess sample is preferably kept to a minimum so that the chemical or biochemical reactions occurring in the reaction mixture are not substantially influenced by the excess sample in the sample addition region 1. Groove depths can range from about 0.01 mm to 0.5 mm or from about 0.05 mm to 0.2 mm. When more than one groove is used for this element, the number of grooves in this element can be between 10 and 500 grooves per cm or from about 20 to 200 grooves per cm. Sample from the sample addition region 1 flows over the grooves 12 by capillary action and then into the distal regions of the device. In a further suitable embodiment, grooves, hereafter termed “fingers” 16, are situated in the wall of the adjacent device regionin fluid contact with the grooves 12 or capillary space of the sample reaction barrier 2. These fingers 16 are typically 0.5 mm to 2 mm wide or 1 mm to 1.5 mm wide and typically 0.1 mm to 1.5 mm in depth or about 0.2 to 1 mm in depth. The fingers 16 aid in the capillary flow of the sample into the device. That is, the fingers allow fluid to move from a capillary where the capillarity is relatively high to a capillary where the capillarity is lower. Thus, the capillary at the sample reaction barrier is generally more narrow and has a greater capillarity than the capillary or space of the reaction chamber. This difference in capillarity can cause the flow of sample or fluid in the device to stop in the sample reaction barrier capillary. Presumably, the fingers break the surface tension of the fluid at the interface of the two capillaries or spaces and thereby cause the fluid to move into a capillary or space of lower capillarity. One can appreciate that the utility of fingers can be extended to any part of the device where fluid must flow from high capillarity to low capillarity. In practice, this is usually when the direction of fluid flow is from a narrow capillary (higher capillarity) to a wider capillary (lower capillarity).

The surfaces of the capillaries and chambers in the device can be generally hydrophilic to allow flow of the sample and reaction mixture through the device. The surface opposing the chambers can be hydrophobic such that the reaction mixture repels this surface. The repulsion of reaction mixture to the surface opposing the chambers forces the reaction mixture, and particularly the protein conjugates, to the surface where optional capture can occur, thus improving the capture efficiency of components of the reaction mixture to a capture zone. The hydrophobic surfaces opposing the diagnostic element can have a tendency to become hydrophilic as the reaction mixture progresses through the diagnostic element because various components which may be present endogenously or exogenously in the sample or reaction mixture, such as, for example, proteins or polymers, bind to the hydrophobic surface. A suitable hydrophobic surface opposing the diagnostic element can be composed of TEFLON®. It is well known to those skilled in the art that TEFLON® surfaces bind proteins poorly. Thus, the TEFLON® surface opposing the chambers would not become as hydrophilic as would surfaces composed of, for example, polystyrene, polyacrylate, polycarbonate and the like, when the reaction mixture flows through the chambers and capillaries of the device.

In another embodiment, the chambers can be hydrophilic but the areas adjacent to the chambers are hydrophobic, such that the reagents of the assay are directed through only the hydrophilic regions of the diagnostic element. One skilled in the art will recognize that various techniques can be used to define a hydrophilic chamber, such as plasma treatment of hydrophobic surfaces using masks which shield the surfaces, except for the chamber, from the treatment or by application of hydrophobic adhesives to hydrophilic surfaces to define a chamber or by the use of viscous hydrophobic compounds, such as an, oil or a grease. In another embodiment, the capillary of the chamber can be formed by ultrasonic welding. The boundaries of the chambers are dictated by the energy directors which are used to form the sonicated weld.

The capillary space can be defined by a variety of ways, for example, machining the surfaces to the appropriate tolerances or using shims between the surfaces. In a suitable embodiment, ultrasonic welding of the surfaces defines the capillary. In this case, the capillary space is defined by the energy directors and the distance between the surfaces is a function of the size of the energy director, the welding energy, the time of energy application and the pressure applied during welding. The surfaces of the chambers can be parallel or non-parallel. In the latter case, the flow rate of the reagents through the chambers will not be uniform throughout the length. The surfaces of the chambers can be made from materials, such as plastics which are capable of being milled or injection molded, for example, polystyrene, polycarbonate, polyacrylate and the like or from surfaces of copper, silver and gold films upon which are adsorbed various long chain alkanethiols as described in Laibinis & Whitesides, J. Am. Chem. Soc. 114, 1990 (1992) and the references therein. In this latter example, the thiol groups which are oriented outward can be used to covalently immobilize proteins, receptors or various molecules or biomolecules which have attached maleimide or alkyl halide groups and which are used to bind components from the reaction mixture.

The top surface of the sample reaction barrier can also be used to immobilize reagents used in the assay process such that the sample flows over the sample reaction barrier, dissolves the reagents and moves into the second device region. The movement of the sample and reagents into the second device region 3 chamber can act as a mixing means.

### Second Device Region

Referring to FIG. 2, the fluid sample moves into a second device region 204 spaced from the sample addition region 201 such that fluid introduced into the sample addition region 201 flows along a flow path to the second device region 204. In certain embodiments, the sample will pass through analyte detection region 203 to reach second device region 204. In alternative embodiments, for example as are depicted in FIG. 3, the analyte detection region 303 may not lie on the same flow path as the one connecting sample addition region 301 and second device region 304. Various reagents of the device, and in particular the labeled conjugates, can be placed in the second device region 204, for example, as dried or lyophilized powders, such that when the fluid sample enters the second device region 204 the reagents quickly reconstitute. The magnetically responsive particles can also be placed in the second device region 204 such that when the fluid sample enters the second device region 3, the reaction mixture can be constituted. In alternative embodiments, for example as are depicted in FIG. 3, the magnetically responsive particles can also be placed in another region of the device, for example in an optional reagent chamber 302.

As previously mentioned, in various embodiments, the population of magnetically responsive particles can be introduced into the assay device prior to, together with, or after addition of the sample fluid to the assay device. The population of magnetically responsive particles can be disposed within the assay device prior to application of said fluid sample. In certain embodiments, the population can be located within any portion of the assay device prior to application of said fluid sample, as they can be transported to the second device region by application of a magnetic field if necessary. This can be referred to as an optional “third device region.” In other embodiments, the population of magnetically responsive particles is disposed in or on the second device region such that their movement by application of a magnetic field is not required to form the reaction mixture. In still other embodiments, the population of magnetically responsive particles is transported to the second device region at least in part by fluid flow occurring upon addition of the sample fluid to the assay device.

Mixing features which mix the reaction mixture can also be incorporated in conjunction with the second device region 3, such as those described in WO92/21434, hereby incorporated by reference. The sample fills the regions of the device to arrive at the second device region due to capillary forces, the force resulting from hydrostatic pressure, by application of force generated via mechanical pumps, electroosmotic pumps, centrifugal force, increased air pressure, or by a combination of two or more such forces.

The volume of the second device region can be any volume which accommodates the reagents and which provides the desired sensitivity of the assay. The shape of the second device region chamber, and/or the chamber or lane extending from the second device region to the location at which magnetic particles are positioned in the device, may be designed such that the movement of the reaction mixture as a result of the movement of magnetic particles in or out of the chamber is reduced or minimized. Upon contacting the second device region with magnetically responsive particles, magnetic force may be used to “mix” the reaction mixture, to improve capture efficiency and reduce assay variability, prior to transport of the magnetically responsive particles to the detection region of the device. A suitable shape of the second device region is shown in FIG. 1 as element 3, however the precise shape is not critical. The width and depth are preferably of mesoscale dimensions, and can range from about 0.01 mm to 10 mm. In certain embodiments, the width and/or depth are between 0.03 mm to 0.6 mm

### Optional Reagent Chambers

Referring to FIG. 3, an optional reagent chamber 302 can be useful for the introduction of additional reagents into the assay process. In general, the optional reagent chamber 302 can be in fluid contact with the sample addition region 301, with a flow path leading to the second device region 304, and/or with a flow path leading to the analyte detection region 303. The flow of the introduced reagent can be controlled by a sample reaction barrier similar to sample reaction barrier described above.

### Analyte Detection Region

Referring to FIG. 2, the analyte detection region 204 can be fluidly connected to the second device region 201, and is preferably formed by opposing surfaces which are a capillary distance apart through which a fluid sample flows. As discussed herein, fluid flowing through the device fills a flow path through the device, and creates a reaction mixture at second device region 201. This reaction mixture is contacted with magnetically responsive particles to capture for detection labeled conjugates corresponding to one or more analytes of interest. Following the contact of the labeled conjugates with the magnetically responsive particles in the second device region 3, a magnetic field is configured to induce movement of the magnetically responsive particle complexes on a path toward the analyte detection region 203. The direction of this movement is configured to be different from, and preferably counter to, the direction of the fluid flow from the sample addition region 201 for at least a portion of the flow. As depicted in FIG. 2, the direction of movement of magnetically responsive particle complexes on a path toward the analyte detection region 203 is counter to the direction that fluid fills the device for its entire distance, while as depicted in FIG. 3B, the direction of movement is counter to the direction that fluid fills the device for only a portion of the distance.

In a sandwich assay, a sandwich complex is formed comprising a first analyte receptor immobilized on the magnetically responsive particles, the analyte, and a second analyte receptor bound to a detectable label (the labeled conjugate). In a competitive assay, detectably labeled analyte (the labeled conjugate) and analyte in the sample compete to form a complex with an analyte receptor immobilized on the magnetically responsive particles, or analyte immobilized on the magnetically responsive particles and analyte in the sample compete to form a complex with detectably labeled analyte receptor (the labeled conjugate). In any case, the complex comprising magnetically responsive particles with labeled conjugate bound thereto is delivered to the analyte detection region for generation of a signal from the detectable label. This description is not meant to be limiting, and other suitable assay formats are well known to those of skill in the art.

Once such a complex is delivered within the analyte detection region, the signal representative of the presence or amount of the target analyte in the test sample can be measured. One skilled in the art can appreciate that various means can be used for the detection of signal in the analyte detection region. Exemplary types of optical detection means include, but are not limited to visual and instrumental means, such as spectrophotometric and reflectance methods by a CCD camera, a fluorometer, or a spectrophotometer. Other means of detection known to those skilled in the art can be employed. In detecting an optical label, the analyte detection region can be interrogated with a light source that illuminates the region with an appropriate wavelength for the label being employed, and an optical detector can be positioned to receive transmitted, reflected, or emitted light, depending on the detection method.

While the magnetically responsive particles delivered to the analyte detection region will often be interrogated en masse in order to detect the labeled species bound thereto, in certain embodiments, the analyte detection region can be configured to provide a “flow cell” through which magnetically responsive particles pass in a single file. As each particle passes, the individual particle can be interrogated with a light source, and transmitted, reflected, or emitted light from that individual particle can be detected in a manner akin to flow cytometry. By using a plurality of different detectable particles, each corresponding to a different analyte, such a flow cell arrangement can be advantageous when measuring the presence or amount of multiple analytes. Typical multiplexed systems are based on a technology that uses multiple (up to 100 or more) color-coded particle sets, each of which can be conjugated with a different specific reactant (e.g.; an antibody to a particular antigen). If 100 different particle sets are use, then 100 different species can be simultaneously measured in a single tube or microplate well. Immobilized “bead-bound” capture molecules react with a reaction partner (analyte) in solution. A reporter molecule, specific for the analyte, is used to quantify the interaction (e.g., a second antibody forming a sandwich pair, conjugated to a detectable label). The individual particles are interrogated one at a time, and each particle in a set is identified by its spectral signature. The attendant reporter molecule signal from each reaction is simultaneously quantified.

Alternative methods to multiplex assays can include the use of different detectable label conjugates corresponding to each analyte of interest. These different detectable label conjugates can comprise, for example, an antibody that binds an analyte of interest coupled to a unique (for the analyte) label that is distinguishable from labels used for the other analytes. Optical labels may be distinguished by various spectral properties including absorbance or emission wavelength, fluorescence lifetime, etc. This approach may be combined with the color-coded particle sets described above to further extend the number of analytes that may be distinguished. For example, a combination of 2 distinguishable particles and 2 distinguishable detectable label conjugates can permit one to distinguish signals related to 4 different assays.

The foregoing is described in terms of optically detectable labels, but the skilled artisan will understand that numerous other detection modalities may be employed, depending on the nature of the label. Detection modalities include amperometric, conductimetric, potentiometric, impedimetric, acoustic, fluorescence, reflectance, luminescence, electrochemical luminescence (ECL), interferometric, and surface plasmon resonance (SPR) methods. This list is not meant to be limiting.

In various embodiments, the volume of the analyte detection region can be similar to or different from that of second device region. The shape of the analyte detection region chamber, and/or the chamber or lane extending from the second device region to the analyte detection region, may be designed such that the movement of the reaction mixture as a result of the movement of magnetic particles in or out of the chamber is reduced or minimized. The analyte detection region is preferably substantially flattened, and the width and depth of the analyte detection region are each preferably of mesoscale dimensions, and can range from about 0.05 mm to 10 mm or from about 0.1 mm to 0.6 mm. In preferred embodiments one dimension (depth or width) is from 0.01 to 10 mm and preferably 0.1 to 0.5 mm, while the other dimension (depth or width) is from 0.25 to 2 mm and preferably 0.5 to 1 mm.

The surfaces of the analyte detection region, like the other components of the device, can optionally be smooth, grooved, or grooved and smooth. Various textured surfaces can also be employed, alone or in combination with smooth or grooved surfaces. For example, surfaces composed of posts, grooves, pyramids, and the like referred to as protrusions; or holes, slots, waffled patterns and the like, referred to as depressions can be utilized. The surface can comprise texture structures that comprise the form of diamonds, hexagons, octagons, rectangles, squares, circles, semicircles, triangles or ellipses. The textured surface can comprise texture structures in geometries ordered in rows, staggered or totally random; different geometries can be combined to yield the desired surface characteristics. Typically, the depressions or protrusions of the textured surface can range from about 1 nm to 0.5 mm or from about 10 nm to 0.3 mm; the distance between the various depressions or protrusions can range from about 1 nm to 0.5 mm or from about 2 nm to 0.3 mm. The positioning of the grooves can be perpendicular to the flow of the reaction mixture so that the flow of the reaction mixture through the analyte detection region occurs in an organized manner with a distinct, straight front dictated by the grooves in the capillary space.

In certain embodiments, one or more analyte detection region chamber(s) can be comprised in a device. As depicted in FIG. 4, a fluid sample can be applied to the device at a common sample addition region, depicted as elements 401 and 404, and then separated into two or more different flow paths, with separate second device regions 403 and/or one or more second device regions 408. An optional blood filtering element, depicted as element 405 of FIG. 4 may be included as discussed above. Analyte detection regions 402 and 406 may be provided in different chambers of the device. In certain embodiments, the two or more second device regions can be provided, for example as parallel paths, to provide multiplexed assays (that is, detection of a plurality of analytes from a single sample. In addition, distinct colorimetric or fluorometric labels may also be employed in order to provide multiplexed assays along a single flow path. Various combinations of detection modes are possible and known by those skilled in the art. While the various flow paths of such devices are depicted in “parallel,” the skilled artisan could readily prepare other geometric configurations in one, two and three dimensions and direct the movement of the magnetically responsive particles to a detection region at a velocity different from the fluid flow. The regions can be separate chambers or can be device surfaces that do not delimit a chamber.

As depicted in FIG. 3B, the analyte detection region 303 may not be in the direct flow path from the sample addition region 301 to the second device region 304. In such embodiments, the magnetically responsive particles flow, upon entering a controllable magnetic field, from the second device region 304 counter to the direction of fluid flow through the device for at least a portion of the flow through the device before being moved through a second flow path to one or more analyte detection regions 303.

### Used Reagent Reservoir

Referring to FIG. 1, an optional used reagent reservoir 5 can receive the reaction mixture, other reagents and any excess sample from the upstream regions of the device. The volume of the used reagent reservoir 5 can be at least the volume of the sample and extra reagents which are added to or are in the device. The used reagent reservoir 5 can take many forms using an absorbent, such as a bibulous material of nitrocellulose, porous polyethylene or polypropylene and the like or the used reagent reservoir can be comprised of a series of capillary grooves. In the case of grooves in the used reagent reservoir 5, the capillary grooves can be designed to have different capillary pressures to pull the reagents through the device or to allow the reagents to be received without a capillary pull and prevent the reagents from flowing backwards through the device. The size and quantity of the grooved capillaries determine the volume and capillarity of the used reagent reservoir 5. As depicted in FIG. 4, a fluid sample can be delivered from a plurality of flow paths to a common used reagent reservoir 407.

### Assay Instrument

Another embodiment of the present invention is directed to a system and method for performing measurements on a testing or assay device described above using a measuring assay instrument, such as, for example, a CCD camera, a fluorometer, or a spectrophotometer. The assay device can be used in conjunction with an assay instrument, for example an enhanced fluorometer according to one embodiment of the invention, to achieve a result regarding the concentration or presence of an analyte in a sample. The assay device can include reagents that are necessary for performing an immunological or chemical reaction, such reaction giving rise to a change in fluorescence of the sample that has been treated with the reagents. The reagents can include chemicals, antibodies, peptides, analytes, analyte analogues and these reagents may or may not be coupled to fluorescent labels or to solid phases such as magnetically responsive particles.

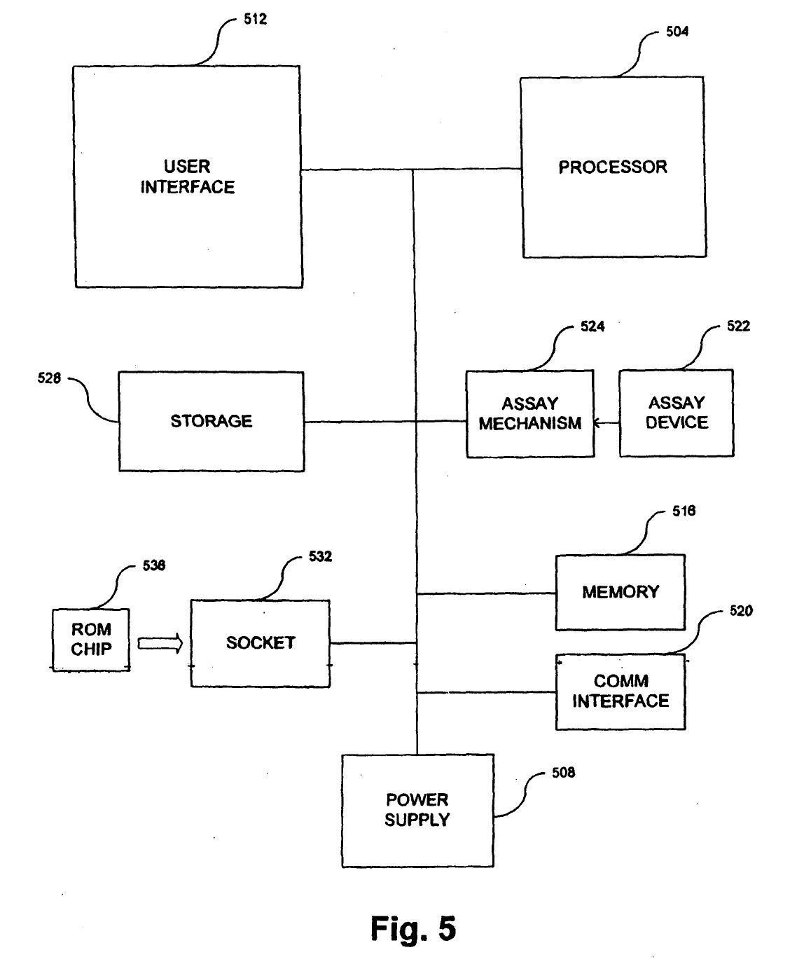

FIG. 5 is a diagram illustrating a functional block diagram of one embodiment of an enhanced assay instrument to that fluorometer described in commonly owned U.S. Pat. No. 6,830,731, which is hereby incorporated by reference in its entirety. FIG. 5 illustrates examples of the functionalities that can be included with the automated fluorometer in terms of one example physical architecture, a central bus structure. The enhanced fluorometer according to the embodiment illustrated in FIG. 5 includes a processor 504, a power supply 508, a user interface 512, a memory 516, a communications interface 520, an assay mechanism 524, an assay device 522, a storage device 528, and removable storage media. In the example illustrated in FIG. 5, the removable media include a ROM chip 536 and socket 532. Any or all of these functionalities can be included with an enhanced fluorometer depending on the particular application. The corresponding description provided in U.S. Pat. No. 6,830,731 (corresponding to FIG. 1) will provide one of ordinary skill in the art the ability to implement any or all of the described functionality using one or more alternative architectures.

Referring to FIG. 5, assay mechanism 524 can be used to perform the fluorometric readings on the assay device in order to test the presence or concentration of one or more analytes. In one embodiment, assay mechanism 524 can be a slide mechanism that is used to accept a small tray-like device, for example, an assay device. Assay mechanism 524 can include the optical components necessary to perform the readings of the reaction complexes as well as a slide on which the assay device slides to position the assay zones in the appropriate location so that fluorescence can be measured in a reproducible manner. In one embodiment, the mechanism is motorized such that the assay device can be automatically loaded and ejected from the fluorometer as well as positioned with respect to the optics and magnets during testing. In this embodiment, the assay device is transported along a path in the slide having magnetic forces to draw the magnetically responsive particle complexes to the analyte detection region 4 for measurement by the fluorescence. The path in which fluorescence measures the detectable labels in the assay device is referred to as the “diagnostic lane” of the device.

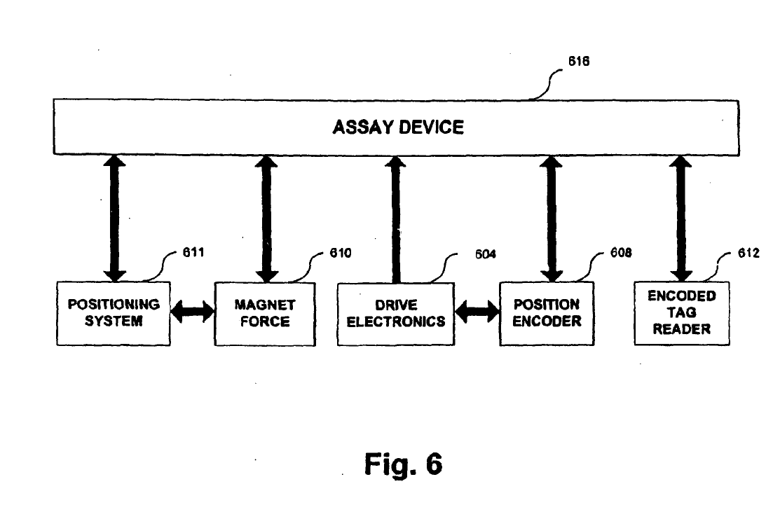

FIG. 6 is a diagram illustrating an example implementation of an assay mechanism or assay device drive, according to one embodiment of the invention. The assay device drive according to the embodiment illustrated includes drive electronics 504, a position encoder 508, a magnetic force 610, and an encoded tag reader 512 such as, for example, a bar code reader. In one embodiment, drive electronics 604 includes a motor to position the assay device and a motor controller to control the motor. A friction drive, belt drive, gear drive, or other mechanism can be used to translate the rotation of the motor into motion of the assay device. Drive electronics 604 are thus used to load and eject the assay device as well as to position the assay device with respect to the optics of the fluorometer, for example, along the diagnostic lane. In this embodiment, the assay device is moved in relation to stationary optics. In alternative embodiments, the optics can be moved instead of, or in addition to, the assay device.

In various suitable embodiments, the assay instrument provides a controllable magnetic field from the magnetic field source. As used herein, the term “controllable magnetic field” refers to a magnetic field that can be applied to the assay device in intensities that vary in space and/or time. The skilled artisan will understand, for example, that the intensity of a magnetic field at a location will fall as the distance between the location and the magnetic field source increases. Thus, the assay instruments of the present invention can provide a controllable magnetic field through the particular shape of the pole faces and/or by providing one or more positioning systems that alter the distance between the magnetic field source and the assay device. In such a case, either the magnetic field source, the assay device, or both can be moved by the positioning system(s). Alternatively, the magnetic field source can be controlled electronically, as in the case of an electromagnet. In still another alternative, a movable shield that alters the intensity of the magnetic field applied to the assay device can be provided as a component of either the assay instrument or the assay device. Other elements that can provide a controllable magnetic field in such instruments will be apparent to those of skill in the art.

The influence of magnetic field 610 is used to move the reaction complex comprised of the labeled conjugates, analyte of interest, and magnetically responsive particles from a first location (i.e. the second device region 3) to a second location (i.e. the analyte detection region 4) in a direction counter to the direction of fluid flow through the device for at least a portion of the path, thus reducing contamination of the signal with unbound label as unbound label flowing with the sample fluid will be directed away from the analyte detection region and eliminating the need for wash steps that are common to many methods that incorporate magnetically responsive solid-phase matrices.

Magnetic field strengths up to about one tesla (ten thousand gauss) can be produced by means of permanent magnets. High field strength permanent magnets are typically made using ferrometallic alloys such as aluminum nickel cobalt, ceramic ferrites such as strontium ferrite, or rare earth alloys such as, neodymium iron boron and samarium cobalt. Field strengths greater than one tesla can be generated by means of electromagnets, including superconducting magnets. Both permanent and electromagnets are readily available in a wide range of sizes and designs. For example, high field strength permanent magnets made from neodymium iron boron are available from Kinetic MicroScience (Los Gatos, Calif.), Neomax America (Santa Clara, Calif.), Dexter Magnetic Technologies Inc. (Fremont, Calif.), and Magnet Sales & Mfg. Co. (Culver City, Calif.). Permanent magnets have a number of advantages over electromagnets, the most obvious of which is they require no external power source, and further includes low cost, portability, and flexibility of design. Electromagnets, on the other hand, have the advantage that their field strength can be controlled by means of the electrical drive current.

As one of skill in the art will appreciate, the net force F_{mag} exerted by a magnetic field B upon a magnetic particle in a carrier fluid is given by:

$${F}_{mag} = \frac{{Δ}_{Z} \cdot {V}_{p}}{{μ}_{0}} \cdot ( ∇ \cdot B ) \cdot B$$

where Vₚ is the particle volume, λ_{χ} is the difference in magnetic susceptibilities between the magnetic particles and the carrier fluid, μ₀ is the permeability of free space, and ∇B is the field gradient. To carry out magnetic separations it is often desirable to maximize the magnetic force acting on the magnetic particles. According to the above relationship, for a given particle size and susceptibility, this is achieved by maximizing the product of the magnetic field strength and field gradient. It can be further appreciated that magnetic particles can become trapped in the region where the magnetic field strength is a maximum; i.e., where the field gradient ∇B is zero. Finally, it should be noted that the force on the magnetic particles is proportional to ΔX·Vₚ. For this reason, particle size and size distribution are important considerations for achieving efficient magnetic separation.

Magnetic particles having diameters greater than about 0.3 μm to about 1 μm can be separated using simple permanent magnets while a reasonable separation rate for smaller particles in the range between tens to hundreds of nanometers may require higher magnetic fields that are only achievable by use of electromagnets, including superconducting magnets. In certain embodiments of the current invention where magnetic particles with diameters in the range of 1-10 μm, and with magnetic susceptibilities of typical commercially available super-paramagnetic beads in the range of 1×10⁻⁴ m³/kg to 2×10⁻⁴ m³/kg are used, field strengths in the range of 0.1-1.0 T (tesla) are suitable.

The function of the magnetic field 610 is to move the magnetically responsive particles from a first location to a second location along the diagnostic lane. The first location can be, for example, second device region 3 and the second location can be detection region 4. The source of the magnetic field can be one of the permanent type magnets or electromagnets previously described. In either case, the proximity and shape of the magnet faces or “poles” will primarily determine the field distribution, and in particular the field intensity and gradient. In a suitable embodiment of the current invention the magnetic field used to influence the motion of the magnetically responsive particles can be shaped such as to concentrate the analytes in one or more particular regions of the diagnostic lane for measurement by an optical element. To accomplish this, various advantageous designs of the magnet pole faces can be employed. The source of magnetic field 610 can be a stationary magnetic field gradient or movable magnetic field trap. In the former case a pair of opposing pole faces with the approximate transverse dimensions of the diagnostic lane and with an appropriately varying gap along the direction diagnostic lane can be configured to effect a monotonically varying magnetic field strength along the desired length of the diagnostic lane. It will also be appreciated that a single pole magnet can be configured to produce a suitable magnetic field trap. The resulting field would then tend to move the magnetically responsive particles from regions of weaker magnetic field toward regions of stronger magnetic field with the particles finally becoming trapped at a point of equilibrium in the region of maximum field strength. A particularly advantageous design would produce a field with an approximately constant magnetic field, field gradient product (B*∇B) over most of the desired length of the diagnostic lane so as to produce a correspondingly constant force on the magnetic particles along this path. The pole faces in the dimension transverse to the lane can also be narrowed and shaped to create a transverse magnetic field gradient having a maximum along the center of the diagnostic lane thereby further concentrating the magnetically responsive particles in this region. The region of maximum magnetic field where the magnetically responsive particles are trapped can approximately correspond to diagnostic region 4. During some portions of the operation of the system it may be necessary to partially or completely remove the magnetic field from the assay device. This can be accomplished by one or more of: (1) in the case of a permanent magnet, moving either the assay device and/or the permanent magnet to a remote location or shielding the magnet from the magnetically responsive particles; (2) in the case of an electromagnet, simply lowering or turning off the drive current.

In an alternative embodiment, magnetically responsive particles can be moved from a first location to a second location by means of one or more magnets of either of the permanent or electromagnet type that creates a more localized magnetic trap and wherein the trap region is moved by translating either or both the magnet and/or the assay device. In this embodiment, the magnet poles can be shaped so as to create a field gradient over dimensions that are only required to include the area of second device region 3. Suitable pole shapes can include, for example, a pair of opposing hemispherical poles with a gap between that accommodates the narrow dimension of the assay device. Other pole shapes are possible and include small diameter permanent magnets and electromagnets with tapered cores. Shaped magnetic poles may be used to produces a desired magnetic field gradient, for example to provide a homogenous region (that is, a region where the magnetic field gradient is 0). Microfabricated magnets incorporated into the microfluidic device can also be used. It will also be appreciated that a single pole magnet can be configured to produce a suitable magnetic field trap. In one embodiment of the magnetic field two magnets can be placed next to the down-lane end of the device and the distance between the magnets can be within the range of 1-25 mm. In another embodiment of the magnetic field two magnets can be placed next to the down-lane end of the device and the distance between the magnets can be within the range of 1-10 mm. In a suitable embodiment of the magnetic field two magnets can be placed next to the down-lane end of the device and the distance between the magnets can be 3 mm. It is also possible to use two or more magnets, such as sawtooth gold wire or mesh gold wire. Various magnets and schemes for applying variable magnetic fields is disclosed in Pamme, N., Lab Chip, 6, 24 (2006), which is herein incorporated by reference.

Magnetic field 610 intensity can be controlled by various means known to the skilled artisan. As can be appreciated, for example, the intensity of a magnetic field at a location will fall as the distance between the location and the magnetic field source increases. Thus, the assay instruments of the present invention can provide a controllable magnetic field by providing one or more positioning systems 611 that alter the distance between the magnetic field source and the assay device. In such a case, either the magnetic field source, the assay device, or both can be moved by a positioning system(s). Alternatively, the magnetic field source can be controlled electronically, as in the case of an electromagnet. In still another alternative, a movable shield that alters the intensity of the magnetic field applied to the assay device can be provided as a component of either the assay instrument or the assay device. Other elements that can provide a controllable magnetic field in such instruments will be apparent to those of skill in the art.

Position encoder 608 is used to determine the position of the assay device within assay device drive. Position encoder 608 can obtain position information from the assay device itself such as, for example, by sensing an encoded label on the assay device. Alternatively, position encoder 608 can determine the position of the assay device based on the rotation of the drive shaft through the motor using well-known encoder techniques. Encoded device reader 612 is used to read the encoded tag provided on the assay device. In one embodiment, encoded tag reader 612 is a bar code reader that reads a bar code label on the assay device. Alternative embodiments can include, for example, a magnetic stripe reader, an inductive reader, or an optical character recognizer. An encoded tag reader 612 senses the encoded tag information from the label on the assay device and provides this information to processor 504 of the assay instrument. The encoded information can include information such as, for example, a patient I.D., an identification of the tests to be performed on the sample, an identification of the sample type, or other appropriate or pertinent information. This information can be used to log the test results as well as to control the type of testing performed or test parameters used.

In one embodiment, drive electronics 604 and position encoder 608 are used to direct the positioning of the assay device into the magnetic field so that the assay device can be tested, as well as to reposition the assay device during testing such that a plurality of regions of the assay device can be tested. This capability to position the assay device such that various portions of the sample can be tested allows enhanced testing algorithms to be utilized to produce improved test results. An example of enhanced testing routines that can be used where different regions of an assay device are tested is fully described in copending patent applications of common assignee, now U.S. Pat. No. 5,763,189, having application Ser. No. 08/311,098, and titled “Fluorescence Energy Transfer and Intramolecular Energy Transfer in Particles Using Novel Compounds” and application Ser. No. 08/409,298 also titled “Fluorescence Energy Transfer and Intramolecular Energy Transfer in Particles Using Novel Compounds,” which are incorporated herein by reference.

### Exemplary Embodiments of the Assay Device and Assay Instrument System

The elements of the device which have been described individually can be assembled in various ways to achieve the desired function. In certain embodiments, one manual action is required to achieve the assay result, for example, adding sample to the device is one step. The devices are generally about 3 cm to 10 cm in length, 1 cm to 4 cm in width and about 1 mm to 4 mm thick. The devices can be up to 5, up to 10 or up to approximately 15 mm thick. Typically, a top member with smooth surfaces is placed onto a bottom member which has a surface onto which are built the elements stated above. The reagents required for performing the assay are immobilized or placed in the respective elements. The surfaces are brought together, a capillary distance apart, and in doing so, the regions of the sample addition region, the sample reaction barrier, the second device region, the analyte diagnostic region, the flow path and the used reagent reservoir are all formed and are capable of functioning together. Also, the surfaces are brought together such that the opposing surfaces touch to form and seal the sample addition region, the analyte detection region, and the used reagent reservoir.

In one embodiment for performing a sandwich assay, the assay is performed by addition of sample to the sample addition region of the device. The sample dissolves and mixes any reagents provided in the sample addition region. The sample moves enters the device and flows down a flow path, filling the analyte detection region and the second device region and connecting them fluidly. The sample dissolves the reagents (i.e. labeled conjugates) present within the device. Magnetically responsive particles comprising receptors for one or more analytes of interest contact all or a portion of the sample, capturing the analyte(s) of interest if present. The labeled conjugates in the second device region in turn contact the population of magnetically responsive particles in the presence of at least a portion of the fluid sample, and sandwich complexes are formed. These magnetically responsive particles are configured to form a complex with the labeled conjugates in an amount related to the presence or amount of the analyte of interest in the reaction mixture. If necessary, the assay device can be introduced into the assay instrument in order to provide a magnetic field configured to induce movement of the magnetically responsive particles on a path to the second device region 3.

Following the formation of the sandwich complexes, the magnetically responsive particles, now bound to labeled conjugates and the target analyte, can be separated from the reaction mixture by applying a magnetic field to the assay device. The assay device is introduced to the assay instrument in order to test the presence or concentration of one or more analytes. Because the magnetically responsive particles form a complex with the labeled conjugates in an amount related to the presence or amount of the analyte of interest, the signal detected can be correlated to the presence or amount of the analyte in accordance with standard receptor binding assay methodology.

In one embodiment for performing a competitive assay, the assay is performed by addition of sample to the sample addition region of the device. The sample dissolves and mixes any reagents provided in the sample addition region. The sample enters the device and flows down a flow path, filling the analyte detection region and the second device region and connecting them fluidly. The sample dissolves the reagents (i.e. labeled conjugates, magnetic particles, etc.) present within the device. Magnetically responsive particles comprising receptors for one or more analytes of interest contact all or a portion of the sample, with the labeled conjugates competing with the analyte(s) of interest if present for binding to the receptors. These magnetically responsive particles are configured to form a complex with the labeled conjugates in an amount inversely related to the presence or amount of the analyte of interest in the reaction mixture. If necessary, the assay device can be introduced into the assay instrument in order to provide a magnetic field configured to induce movement of the magnetically responsive particles on a path to the second device region.

Following the formation of the receptor complexes, the magnetically responsive particles, now bound to labeled conjugates, can be separated from the reaction mixture by applying a magnetic field to the assay device. The assay device is introduced to the assay instrument in order to test the presence or concentration of one or more analytes. Because the magnetically responsive particles form a complex with the labeled conjugates in an amount inversely related to the presence or amount of the analyte of interest, the signal detected can be correlated to the presence or amount of the analyte in accordance with standard receptor binding assay methodology.

In another embodiment for performing a competitive assay, the assay is performed by addition of sample to the sample addition region of the device. The sample dissolves and mixes any reagents provided in the sample addition region. The sample enters the device and flows down a flow path, filling the analyte detection region and the second device region and connecting them fluidly. The sample dissolves the reagents (i.e. labeled antibodies) present within the device. Magnetically responsive particles comprising a molecule that competes with one or more analytes of interest for binding to a receptor contact all or a portion of the sample, with the labeled antibodies. These magnetically responsive particles are configured to form a complex with the labeled antibodies in an amount inversely related to the presence or amount of the analyte of interest in the reaction mixture. If necessary, the assay device can be introduced into the assay instrument in order to provide a magnetic field configured to induce movement of the magnetically responsive particles on a path to the second device region.

Following the formation of the complexes, the magnetically responsive particles, now bound to labeled antibodies, can be separated from the reaction mixture by applying a magnetic field to the assay device. The assay device is introduced to the assay instrument in order to test the presence or concentration of one or more analytes. Because the magnetically responsive particles form a complex with the labeled antibodies in an amount inversely related to the presence or amount of the analyte of interest, the signal detected can be correlated to the presence or amount of the analyte in accordance with standard receptor binding assay methodology.

The assay instrument can automatically load the assay device using drive electronics to accurately position the assay device. Upon introduction to the assay instrument, a position encoder determines the position information from the assay device by sensing an encoded label on the device. The position encoder and drive electronics transport the assay device to a controllable magnetic field that induces movement of the magnetically responsive particle complexes within the assay device on a path into the analyte detection region so that the fluorescence of the labeled analytes can be measured. The velocity of the magnetically responsive particles is different from the flow of the fluid sample from the sample addition region for at least a portion of this path, such that contamination of the signal with unbound labeled conjugates is reduced as the magnetically responsive particles are delivered to the detection region more quickly than the diffusion of unbound label to the detection region can produce a substantial background signal. This can eliminate the need for wash steps that are common to many methods that incorporate magnetically responsive solid-phase matrices. A signal is then detected from labeled conjugates in the analyte detection region.

FIG. 11 depicts one mode in which the devices and instruments of the present invention may be used to perform a sandwich assay. In panel A of the figure, a “lane” 1101 of a device is depicted, where end 1102 is fluidly connected to a sample addition region, and end 1103 represents the distal end of the lane. The capacity of the lane is preferably less than 10 μL, more preferably less than 5 μL, and most preferably about 1-2 μL, and is sufficiently hydrophilic to permit fluid to fill the lane. The lane is permitted to fill with sample, whereupon fluid flow is stopped. This may be accomplished by simply reaching the end of the lane, by incorporating a capillary “gap” at the distal end of the lane, or by applying a hydrophobic material to the surface of the lane that acts as a fluid “stop” (depicted as feature 1107 in the figure). Upon and during filling of the device with sample fluid, the magnetically responsive particles 1104 are being held at the proximal end of the lane by magnetic trap 1105, and the labeled conjugates 1106 are reconstituted to provide a reaction mixture near the distal end of the lane. In panel B of the figure, the magnetic trap 1105 has been used to draw the magnetically responsive particles through the lane. Analyte in the sample is captured by the magnetically responsive particles during this movement. The magnetically responsive particles are delivered to the reaction mixture, where sandwich complexes 1108 are formed. In panel C, the magnetic trap 1105 has been used to deliver the magnetically responsive particles “upstream” to a detection region, where optical source 1108 directs electromagnetic radiation to generate a signal from label bound to the magnetically responsive particles, which is detected by optical detector 1109. The magnetic trap may be removed or shielded at this point, for example should it interfere in generation and detection of a signal.

In various alternative modes, detection of label bound to the magnetically responsive particles may be performed as magnetically responsive particles flow past a detector, rather than performing detection from magnetically responsive particles held in a magnetic trap. This mode may be particularly advantageous for multiplexed detection of multiple analytes through the use of detectably different labels corresponding to the analytes to be detected. In this mode, detection is akin to that used in capillary electrophoresis nucleic acid sequencing devices, in which the 4 bases A, T, G, and C are detected through the use of detectably different labels for the four “Sanger” dideoxy reactions. In certain embodiments of this “flow cell” mode, magnetic force may be used to drive the flow of the magnetically responsive particles through a narrow flow path necessitating flow as individual particles, and a detector may be used to identify the label bound to each individual magnetically responsive particle.

The system and methods of the present invention are suitable for this quantitative assay because of the high efficiency of capture of the analytes via the reagents, for example, the binding of a complex of target ligand and receptor conjugate to an immobilized receptor to the target ligand, and because of the rapid and complete movement of magnetically responsive particles, and thereby the label being detected, counter to the flow of the fluid sample directly to the optical sensor using a controllable magnetic force, particularly in a device having a high aspect ratio as described herein. The system and methods of the present invention are also suitable because the movement of the reaction mixture separating the magnetically responsive particles from the flow of the fluid sample reduces contamination to less than 10%, to less than 1%, to less than 0.1%, to less than 0.01%, to less than 0.001%, or to less than 0.0001% of the total available signal present as detectable label conjugate in the device, as unbound label flowing with the sample fluid is directed away from the analyte detection region(s), thereby eliminating the need for wash steps that are common to many methods that incorporate magnetically responsive solid-phase matrices. Preferably, the background contamination of the assay signal is not appreciable, meaning that it does not contribute to the assay signal being detected.

### Examples

The following examples serve to illustrate certain embodiments of the present invention. These examples are in no way intended to limit the scope of the invention.

### Example 1

### Detection of BNP Antigen Using Stationary Magnetic Field Gradient

Preparation of Devices with Microfluidic Channel

The following describes a method and device configured to perform a sandwich assay to detect BNP. While described in terms of BNP as an analyte and a sandwich format, the skilled artisan will understand that the following methods may be modified to perform assays for analytes generally using a variety of assay formats as described above.

The devices used in these examples were made from parts used in the manufacture of the Biosite TRIAGE® assay devices and modified as discussed below. Only details of the devices pertinent to the examples will be given here. The devices comprise a NAS 60 plastic base with nominal dimensions 100 mm×35 mm and a lid, also made from NAS 60 plastic, which is attached to the base via ultrasonic welding. The bases have a topology that consists of shallow ridges and grooves, the most important aspect of which is a lane located along the long axis of the base. When the lid is welded to the base, a channel is formed in the lane area 30-60 mm long, 2 mm wide and 30-50 microns deep. In the present example, the bases and lids were cut so that that length of the device was 20 mm while the length of the lid, and hence the channel, was 18 mm. In the following, one end of the lane shall be referred to as the downward end or down-lane and the other end shall be referred to as the upward end or up-lane.

The steps used to create the modified devices were as follows: 1. Drill a hole in the lid with a #58 drill bit such that the hole was centered over the lane and was 1.5 mm from the downward end. 2. Cut the lid so that it covers an 18 mm length on the final panel. 3. Spray the cleaned base with a mist of casein solution (1 mg/mL) using an EFD spray system (EFD, Inc., East Providence, R.I.). 4. Apply hydrophobic ink to the edges of the lane in order to retain the plasma within the lane and prevent it from flowing along the edges. 5. Spray the lid with lid spray (50% ethanol, 0.025wt % PEG). 6. With a Pipetman, spot 0.2 μl of the primary antibody conjugate, in this case an anti-BNP primary antibody-conjugated fluorescent energy transfer latex (“BNP-FETL”), on the lane 6 mm from the downward end of the lane. 6. Sonically weld the lid to the base. 8. Remove both ends of the device with an end mill to create a 20 mm long panel. Note that the downward ends of the channels were welded closed creating the need for the hole in the lid to allow fluid flow. On the upward end, the lid was shorter than the base by 2 mm resulting in a ledge were fluid could be dispensed.

### Detection of Fluorescence Signal

A fluorescent signal was generated and detected by scanning the devices under an optic block. During the scan, the device was moved at a velocity of about 6 mm/sec with a Parker stage (2-axis Compumotor indexer AT6200 and Parker linear stages P/N 106006BTEP by Parker Hannifin Corp., 5500 Business Park Dr., Rohnert Park, Calif. 94928). The optic block was taken from a Biosite TRIAGE® fluorescence meter and consisted of a 670 nm laser, optics and filters for confocal detection of the 760 nm fluorescent light, a photodiode and signal amplification circuit. The Voltage signal from the photodiode circuit was digitized with a National Instruments Ni-DAQ PCI-MIO-16XE-50 data acquisition card. The software for controlling the Parker stage and data acquisition were written in house in Microsoft Visual Basic 6.

### Materials and Methods for Magnetic Bead—BNP Assay

### 1. Bind Magnetic Dynabeads to BNP Antibody.

The secondary BNP antibodies were coupled to paramagnetic Dynabeads using the following procedure. First, 1.06 mL of BNP antibody solution (as an F(ab′)₂, 10 mg/mL was added to 0.94 mL 50/10/150 buffer (the buffer was at pH 7.0 and contained 50 mM potassium phosphate, 10 mM boric acid and 150 mM sodium chloride). The BNP Ab was reduced with 5 mM DTT (DTT, Product #20290, Pierce Biotechnology, Inc.) and stirred at room temp for 30 min. The resulting solution was purified by size-exclusion chromatography using a 1.5 cm diameter column and 40 mL G-50 gel. 1.5 mL magnetic beads (Dynabeads M-270 Amine, Prod #143.07, Dynal Biotech ASA, Oslo, Norway) were washed three times in 50/10/150 buffer. The beads were reconstituted to 4.5 mL to make a 1% mixture. SMCC (Product #22360, Pierce Biotechnology, Inc.) dissolved in DMF (Product #22705-6, Sigma-Aldrich, Co.) (10 mg/mL SMCC (DMF)) was added to the beads to a final concentration of 0.5 mM and agitated at room temp for 2 h. The reaction was quenched with 20 mM Taurine (Product #T-0625, Sigma Chemical Corp.) for 30 min at room temp. The beads were washed three times with 50/10/150 buffer and reconstituted in 50/10/150 to make a 2% mixture. 2.1 mL of this mixture was added to the purified reduced BNP Ab-solution and agitated at 4 C over night. The reaction was quenched with 1 mM BME (Product #M6250, Sigma-Aldrich Co.) for 30 min at room temp and then with 1 mM NEM (Product #128287, Sigma-Aldrich, Co.) for 15 mM at room temp. The beads were washed three times with 50/10/150 buffer and reconstituted in 1.0 mL in 50/10/150 buffer to make a 2% by weight mixture.2. Incubate Dynabead-BNP antibody complex with BNP standard.

5 μL Dynabead-BNP antibody suspension was mixed with 15 μL BNP standard calibration solution (Biosite Incorporated) in an Eppendorf tube. The mixture was vortexed and incubated at room temperature for 20 minutes. The Eppendorf tube was then stored on ice until used.

In order to test the assay response, different BNP calibration solutions were used in for different experiments. The tested BNP concentrations, when mixed with the bead suspension, ranged from 0 to 2303 pg/ml.

### Observing the Assay Response

The suspension of Dynabead-BNP antibody+antigen complex was pipeted unto the device at the up-lane end. The suspension was observed to flow down the lane by capillary action and reached the BNP-FETL spot in less than 10 seconds. At this point, the Dynabeads were spread evenly across the lane. Then a magnet (neodymium iron boron magnet, 11 mm×11 mm, from Kinetic MicroScience, 19395 Montevina Road, Los Gatos, Calif. 95033, Scitoys Levitaion Bundle #2) was placed next to the down-lane end of the device for one minute. One magnetic pole pointed toward the lane creating a magnet field gradient in the direction of the magnet. The magnet drew the paramagnetic beads to the BNP-FETL spot area. The remainder of the lane was observed to be clear of the brown Dynabeads within 30 seconds.

The magnet was then removed and the device was scanned for fluorescence signal, an operation requiring approximately one minute. Then the magnet was placed at the up-lane and of the device for one minute to draw the paramagnetic beads away from the BNP-FETL spot. The beads were observed to move up-lane and form a cluster at the edge of the lid.

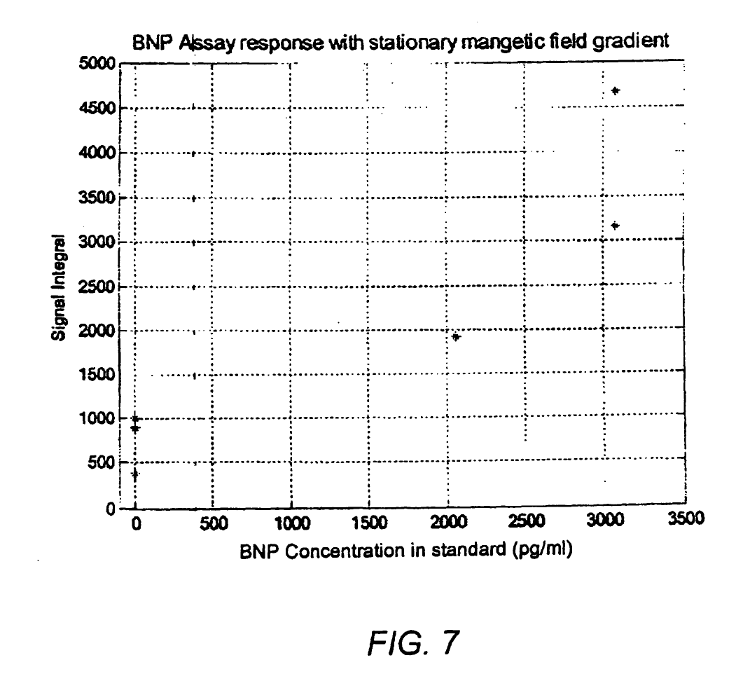

After removing the magnet, the device was again scanned for fluorescence signal. A new signal peak was observed in the location of the beads. FIG. 7 shows a graph of the strength of this signal vs. BNP concentration in the calibration solution used above. The signal strength correlated with the concentration of BNP.

### Example 2

### Detection of BNP Antigen Using Moving Magnetic Field Trap

Preparation of Devices with Microfluidic Channel

As in Example I, the devices used in this example were made from parts used in the manufacture of Biosite's TRIAGE® devices and modified as discussed below. In the present example, only the lids were cut to allow a platform for dispensing solution unto the base. The shape of the base was not modified and, as such, each base was 100 mm long with one rounded and one flat end. In the following, the rounded end shall be referred to as the “nose” or down-lane end and the flat end shall be referred to as the upward end or up-lane.

The steps used to create the modified devices were as follows, with reference to the various reagents described in Example 1: 1. Cut the flat end off of the lids so that the length of the lid was 70 mm from nose to cut end. 2. Spray the cleaned base with casein solution and apply ink to the edges of the lane. 3. Spray the lid with lid spray. 4. Apply grease pencil across the lane of the device with grease spot centered 54 mm from the nose. The purpose of the grease pencil was to stop the flow of fluid in the lane. Enough grease was applied to completely cover the lane while still allowing an air gap between the grease and lid. 5. With a Pipetman, spot approximately 0.02 μl of BNP-FETL on the lane 50 mm from the nose of the device. 6. Sonically weld the lid to the base. The distance between the grease pencil spot and the cut end of the lid, and hence, the length of the channel, was 34 mm.

### Detection of Fluorescence Signal

Fluorescent signal was detected in the same manner as in Example 1 above.

### Materials and Methods for Magnetic Bead—BNP Assay

The assay was created in the same manner as in Example 1 above.

### The Magnetic Trap

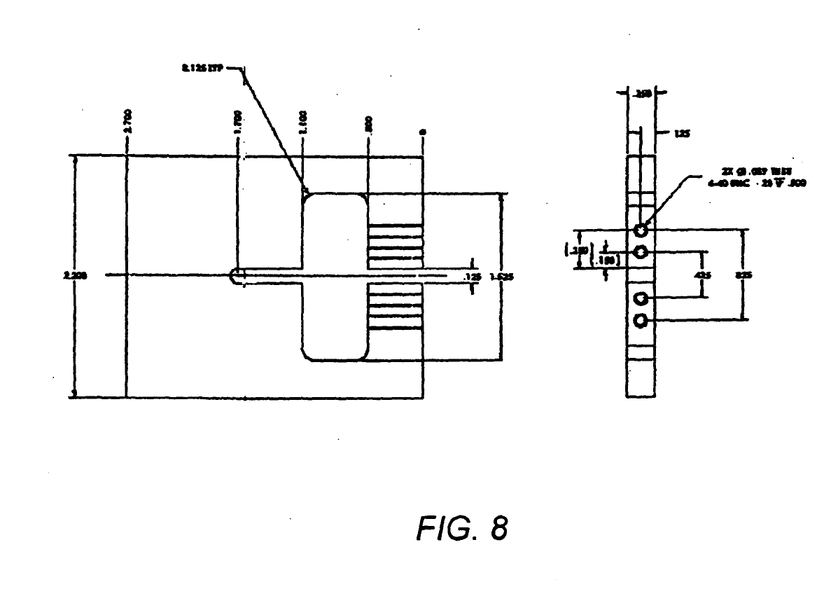

The magnetic trap was created by placing two cube shaped magnets (neodymium iron boron magnet, 11 mm×11 mm, from Kinetic MicroScience, 19395 Montevina Road, Los Gatos, Calif. 95033, Scitoys Levitaion Bundle #2) close to each other with the north pole of one magnet pointing towards the south pole of the other magnet. The distance between the magnets was 3 mm. The magnets were held in place with an aluminum fixture show in FIG. 8. The magnets created a volume with high magnetic field gradient between them, which attracted the paramagnetic beads. In the following, this will be referred to as the “magnetic trap”.

By placing the assay device between the two magnets with part of the lane located in the region of high field gradient, it was possible to attract the beads to this location of the lane. Then by moving the device slowly through the magnets, it was possible to sweep the magnetic trap along the length of the lane. The beads became caught in the trap and could then be moved to any desired location in the lane. If the device was moved quickly away from the magnets, the beads would not be able to move with the magnetic trap and would remain deposited on the lane. By varying the speed at which the device was pulled away from the magnetic trap, it was even possible to spread the beads out along a predetermined length in the lane as they were removed from the trap.

In practice, the device was attached to the same Parker linear stage as used for the detection of fluorescence signal. The device was held with an adjustable lens holder (Melles Griot, 55 Science Parkway, Rochester, N.Y. 14620, part number 07LHA001), and the lens holder was attached to the stage using optical hardware (Thorlabs, Inc., 435 Route 206, Newton, N.J. 07860, part numbers TR4, and RA90). The fixture holding the magnets remained stationary and was attached to an optical bench (Thorlabs, part numbers TR12, TR4, RA90, and MB1824).

### Observing the Assay Response

The suspension of Dynabead-BNP anitbody+antigen complex was pipeted unto the device at the edge of the lid. The suspension was observed to flow down the lane by capillary action and reached the BNP-FETL spot in 10-20 seconds. At this point, the Dynabeads were spread evenly across the lane. The device was then inserted into the adjustable lens holder with the magnetic trap several mm away. A program was written to control the stage in a predetermined series of moves using Visual Basic 6. Table 1 shows the series. Position is the distance between the center of the magnetic trap and the nose of the device. Velocity is the speed of the device when moving the magnets to be at that position.

TABLE 1

| Move Number | Position | Velocity | Delay | |
|---|---|---|---|---|
| 1 | 21 mm | 20 mm/sec | 0 | |
| 2 | 56 mm | 1.5 mm/sec | 0 | |
| 3 | 19 mm | 40 mm/sec | 120 | sec |
| 4 | 47 mm | 40 mm/sec | 0 | |
| 5 | 30 mm | 1.5 mm/sec | 0 | |
| 6 | −21 mm | 70 mm/sec | 0 | |

After move 1, the magnets were located over the end of the lid (the up-lane end of the channel). During move 2, the magnetic beads were collected by the trap and moved to the BNP-FETL spot. In move 3, the beads were spread out over a 2 mm section of the lane in the FETL region as the magnets moved away. After move number 3 there was a delay of 120 seconds to allow the magnetic beads to incubate with the BNP-FETL particles without the influence of magnet fields. In moves 4 and 5, the magnets returned to and collected the magnetic beads once again and then moved them to a location 25 mm away from the BNP-FETL spot. During move 6, the device was moved out of the magnets with a velocity sufficient to have little impact on the bead location.

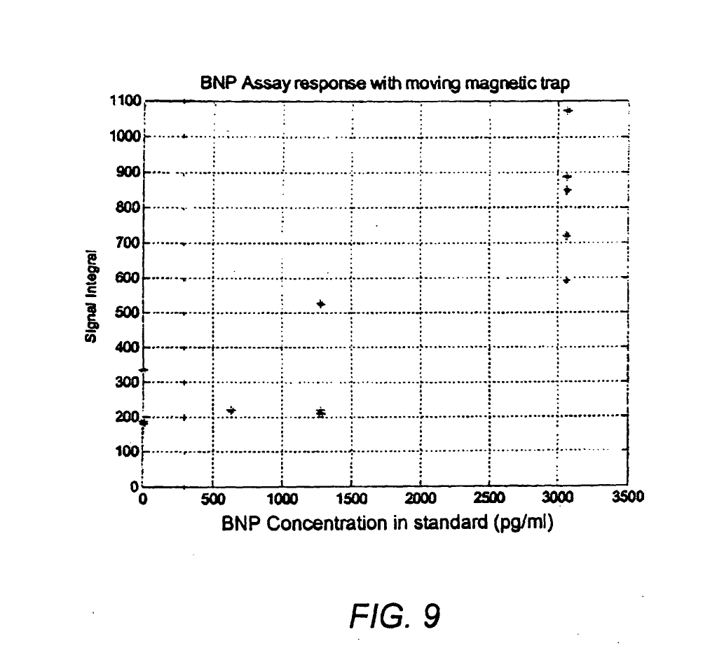

After removing the device from the magnetic trap, the device was scanned for fluorescence signal. A signal peak was observed in the location of the beads. FIG. 9 shows a graph of the signal strength vs. BNP concentration. As in example I above, the signal strength correlated with the concentration of BNP.

Observing the Assay Response v. Time

After moving the beads away from the BNP-FETL spot and scanning them for fluorescent signal, it was possible to move them back to the BNP-FETL spot allowing the beads and BNP-FETL particles to incubate further. Then, after this second incubation period, the beads could be moved away from the BNP-FETL spot and scanned again. In this manner the magnetic trap was used to alternately move the beads to and from the BNP-FETL spot, for incubation and signal observation. The total incubation times were recorded and plotted vs. fluorescent signal strength. If BNP antigen was present in the plasma, the signal was observed to increase over time.

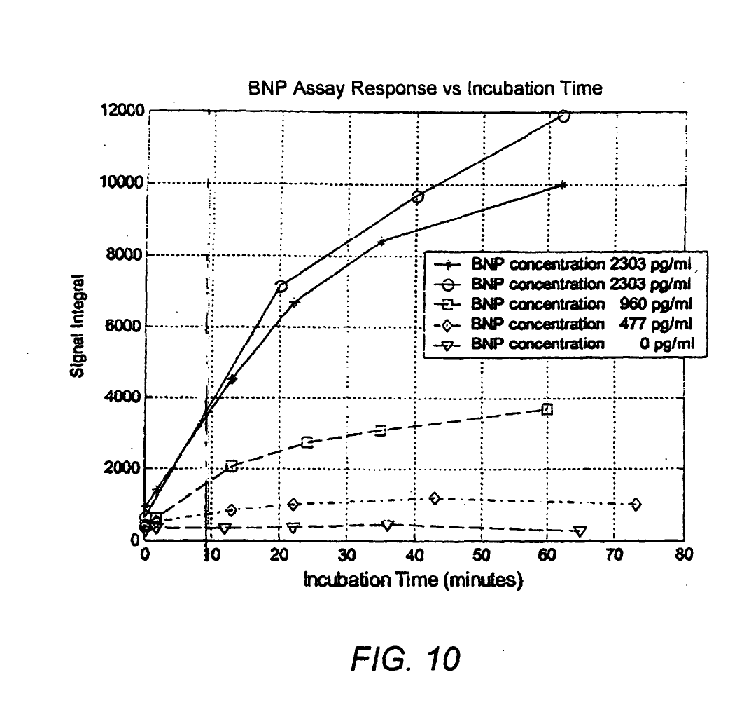

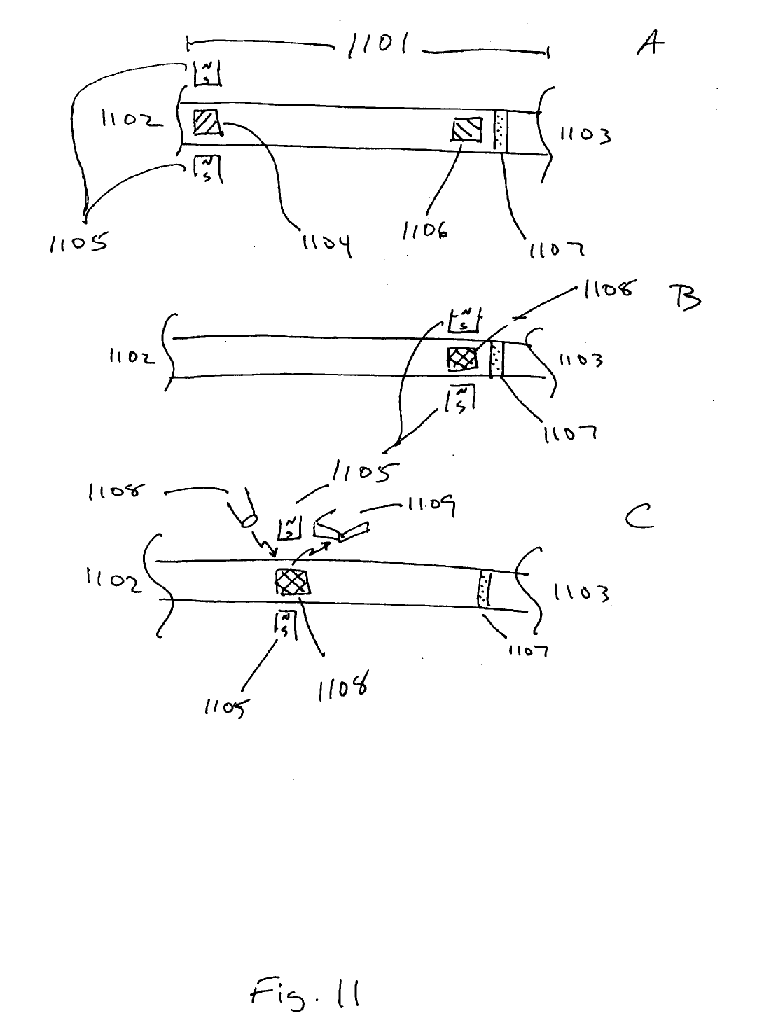

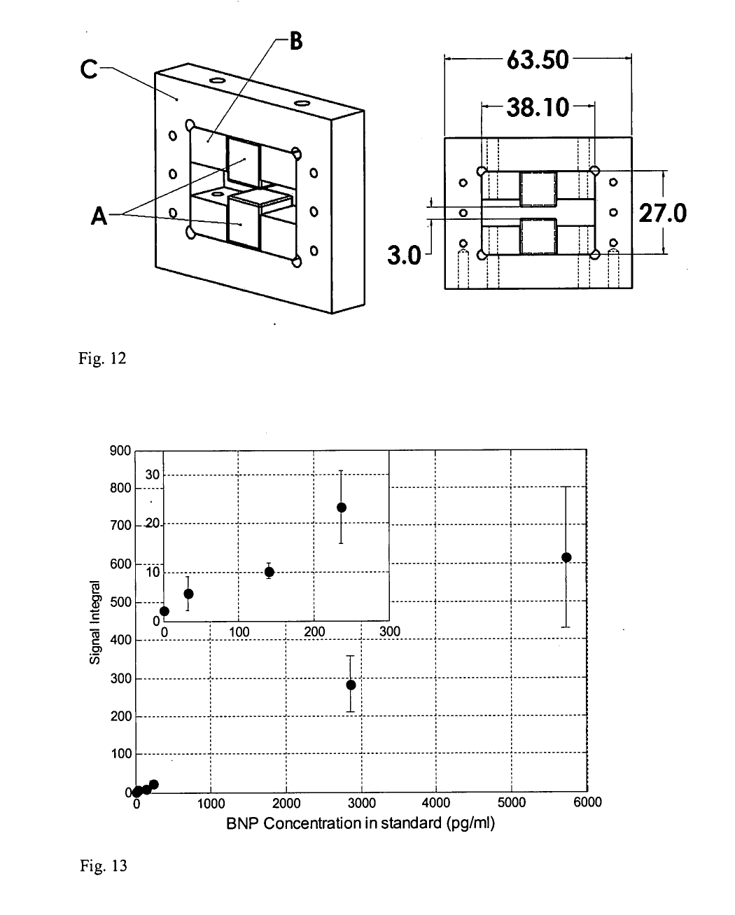

FIG. 10 shows fluorescent signal response verses total incubation time for four different concentrations of BNP antigen. At all incubation times, the signal strength was correlated to BNP concentration.

### Example 3

### Multiplexed Detection of Analytes

For many applications, such as biomarker screening, it is desirable to have multiple assays measured simultaneously. Measuring tens or hundreds of markers in the same sample volume has benefits in throughput and volume per assay. This example describes how one would measure tens or hundreds of assays using microliter scale sample volume. The previous examples provide details on the reagents and procedures used for this application.

In this example, the magnetic beads for a particular analyte carry a unique label. This enables separation of the assay signals at the detector. Examples of unique labels include fluorescent dyes or optical barcoding techniques, such as the Luminex xMAP technology; Bio-Rad BioPlex 2200 system reagents; and Oxonica Inc.'s NANOBARCODE® technology, which comprises multimetal microrods. In particular, the BioPlex system uses labeled magnetic beads. While the other systems do not, introducing magnetic properties to such labels is straightforward to one of skill in the art.

The assay is run in the same manner as the previous examples, i.e. beads are moved into the device region comprising the labeled reagent(s), incubated, then move counter to the direction of flow to move into a clean background at the detection zone. After the beads are at the detection zone, they are then measured one at a time. This is where this technique deviates from the previous examples.

The beads are moved through a measurement region that is configured to enable interrogation of individual beads. This can be achieved by arranging a narrow channel and pulling the beads through via magnet field gradients. To prevent clogging, some form of mixing may be required, either from vibrational souces such as piezo elements, or magnetically stirring the beads. This narrow channel replaces the flow cell use in systems like Luminex® 200™ system or the BioPlex 2200 system. The detection arrangement is similar however. A laser excites the fluorescent dyes in the magnetic particle, and the fluorescent signal is detected and analyzed, identifying the particle, and thus identifying the analyte being measured. A second laser (or other source of light) excites the label attached to the antibodies captured on the magnetic particle, and the resulting fluorescence will be detected. This second signal is related to the amount of analyte present on the bead, and therefore the presence or concentration of analyte in the sample. A statistical sampling of each assay type may improve the precision of the measurement.

### Example 4

### Detection of BNP Antigen Using Moving Magnetic Field Trap

### Preparation of Antibody Conjugate (68 nm Fluorescence Energy Transfer Latex (FETL) Particles Conjugated to Anti BNP Antibody)

FETL-antibody conjugates were prepared essentially as described in U.S. Pat. No. 6,887,952, which is hereby incorporated in its entirety, including all tables, figures, and claims. Carboxyl modified polystyrene latex particles (Interfacial Dynamics, 0.068 μm) are dye loaded by treatment with a solution of fluorescence energy transfer donor and acceptor fluorescent dyes (see, e.g., U.S. Pat. Nos. 5,673,189; 6,238,931; and 6,251,687, each of which is hereby incorporated in its entirety, including all tables, figures, and claims) dissolved in an organic solvent. These fluorescence energy transfer latex particles are referred to as “FETLs.”

For coupling to antibody, a bifunctional crosslinker is used to add a linker arm to the FETL via covalent coupling to FETL carboxyl groups. Thiol groups are then generated from the linker arm under basic conditions. Bovine serum albumin containing maleimide groups is then coupled to the FETL by reaction of the maleimide groups with the thiols on the particle to generate FETL-BSA. FETL-BSA is further treated with a hetero-bifunctional linker to introduce reactive maleimide groups. Excess unreacted linker is removed by column purification. Recombinant antibody to BNP is thiolated by treatment with a second hetero-bifunctional linker. Column purified thiol-activated antibody is then coupled to FETL-BSA-maleimide. Un-reacted thiol and maleimides groups are then blocked and antibody coated FETL is column purified. The particles are stored frozen at −70° C.

Preparation of Devices with Microfluidic Channel

The devices used in this example were made from parts similar to those used in the manufacture of Biosite's TRIAGE® devices with modifications as discussed below. The shape of the base was not modified and, as such, each base was 100 mm long with one rounded and one flat end. In the following, the rounded end shall be referred to as the “nose” or down-lane end and the flat end shall be referred to as the upward end or up-lane. The lane was 1 mm wide by 40 mm long and 30-50 microns deep. The down-lane end of the lane was terminated by a hole in the base which served to stop the flow of fluid. The upward end of the lane had a section 2 mm wide which served as the sample entry location. A hole in the lid directly above the 2 mm wide section of lane served as the sample entry port.

The steps used to create the modified devices were as follows, with reference to the various reagents described in Example 1: 1. Spray the cleaned base with casein solution. 2. Spray the lid with lid spray. 3. With a Hamilton syringe, spot 0.3 μL of 68 nm diameter BNP-FETL on the upward end of the lane. The FETL spots were 5 mm long and centered approximately 3 mm from the hole in the base. 4. Weld the lid to the base.

### Detection of Fluorescence Signal

In this example, the fluorescent signal was detected using an epifluorescence detection scheme. In this configuration the excitation source, a 670 nm laser with the beam parallel to the sample plane, is collimated via an aspheric lens, passed through a 690 nm short pass filter, and then spread into a line using a cylindrical lens. The light is then reflected at a right angle using a dichroic mirror, through an aspheric lens, and the line is focused onto the sample. The resulting fluorescence is collected back through the objective lens, and passes through the dichroic, followed by a fluorescence emission filter at 760 nm, imaged using an aspheric lens, and detected on a photodiode array. The resulting current on the photodiode array is subsequently amplified and digitized.

### Materials and Methods for Magnetic Bead—BNP Assay

Anti-BNP antibodies were coupled to paramangetic Dynabeads (Product #142-04, M-280 Tosyl activated magnetic beads, Invitrogen (Dynal), 1600 Faraday Avenue, PO Box 6482, Carlsbad, Calif. 92008) using the follow procedure. First, the beads were vortexed for 1 min to disperse clumps. Then, the beads were washed 2 times with borate buffer (0.1M boric acid, pH 9.5). Washing consisted of pulling beads to the side of the tube using a magnet and pipetting out the remaining supernatant solution. The beads were then diluted to 0.2% w:v in borate buffer, and NEM (N-ethylmaleimide) blocked HSA (human serum albumin) was added to achieve a final concentration of 1% w:v HSA. The resulting bead mix was vortexed vigorously and probe sonicated in a glass vial. Then the beads were rocked for at least 16 hours at room temperature with vortexing every 2-3 hours during the incubation period. After the 16 hour incubation, the beads were washed with 50/10/150 buffer (pH 7.0 buffer with 50 mM potassium phosphate, 10 mM boric acid and 150 mM sodium chloride) 3 times.

SMCC (Succinimidyl 4-[N-maleimidomethyl]cyclohexane-1-carboxylate) (Product #22360, Pierce Biotechnology, Inc., P.O. Box 117, Rockford, Ill. 61105) was dissolved separately at 20 mg/mL in acetonitrile. Then the beads were added to sufficiently dissolved SMCC to reach a final concentration of 1 mM. and incubated on a rocker for 2 hours. This reaction was quenched by adding 20 mM taurine (Product #T-0625, Sigma Chemical, Corp., St. Louis, Mo., 63178-9916) (final concentration) for 15 minutes followed by a bead wash 4 times with 10/2/200 buffer (10 mM potassium phosphate, 2 mM potassium borate, 200 mM NaSCN, pH 7.0). Finally, sufficient EDTA (ethylenediamine tetraacetic acid) was added to the beads to reach 0.1 mM final concentration.

During the 2 hour SMCC reaction, a separate solution was prepared of SPDP (N-Succinimidyl 3-(2-pyridyldithio)-propionate) (Product #P3415, Sigma-Aldrich, Co, 3050 Spruce St., St. Louis, Mo. 63103) linked HSA by reacting 5 mg/mL of HSA with 1 mM SPDP (final concentration; Stock SPDP is 40 mM in acetonitrile). After a one hour incubation, the reaction was quenched with 20 mM taurine for 30 minutes and column purified using a G-50 column. The SPDP linked HSA and BNP antibody were reduced with 2 mM of DTT (Dithiothreitol) (Product #20291, Pierce Biotechnology, Inc., P.O. Box 117, Rockford, Ill. 61105) for 30 minutes and purified with DG-10 desalting column (Bio-Rad, Hercules, Calif.).

Finally, 0.64 mg/mL of reduced HSA-SPDP and 0.47 mg/mL of reduced BNP antibody were added to 1 mL of 1% magnetic particle and allowed to react for 15 hours. This reaction was quenched with 2 mM of methoxy-PEG-sulfhydryl for 30 minutes, and subsequently quenched with 6 mM of n-hydroxy ethylmaleimide (Product #0-268-116, Organix, Woburn, Mass., 01801) for 30 minutes. The beads were then washed beads 3 times with 50/10/150 buffer.

In some cases, an additional step of casein blocking the beads was performed. 100 μL bead suspension was mixed with 1 ml casein solution and 35 mL of 1M sodium ascorbate solution, and the resulting mixture was placed on a rocker for two hours. The beads were then washed twice in 50/10/150 buffer. Additional components were then added: sodium azide (Product #S2271-1, Fisher Scientific, 81 Wyman Street, Waltham, Mass. 02454) at 1 mg/ml; bovine serum albumin (Product #100 350, Roche Diagnostics/Boehringer Mannheim, 9115 Hague Road, Indianapolis, Ind. 46250) at 10 mg/ml; and sodium ascorbate at 20 mM. The final volume of the bead suspension was 100 μL.

Either 2.5 μL or 5 μL of the Dynabead-BNP antibody suspension was mixed with 30 μL BNP standard calibration solution in a microcentrifuge tube. The mixture was vortexed and incubated at room temperature for 20 minutes. The microcentrifuge tube was then stored on ice until used.

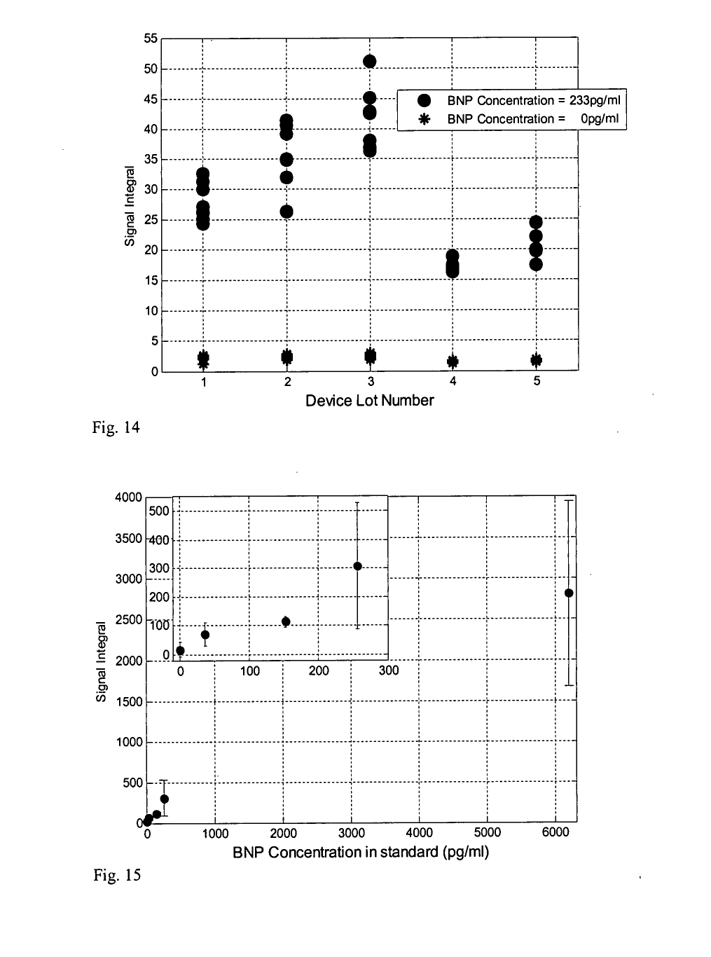

In order to test the assay response, different BNP calibration solutions were used in for different devices. The concentrations of BNP, when mixed with the magnetic particle suspension, ranged from 0 to 6000 pg/ml. As in example 2, a magnetic trap was created by placing two cube shaped magnets (neodymium iron boron magnet, 11 mm×11 mm, from Kinetic MicroScience, 19395 Montevina Road, Los Gatos, Calif. 95033, Scitoys Levitation Bundle #2) close to each other with the north pole of one magnet pointing towards the south pole of the other magnet. The distance between the magnets was 3 mm. In this example, the magnets were held in place using an iron bridge, as shown in FIG. 14. The iron in the bridge served to increase the magnet field in the “magnetic trap” and decrease the stray fields. The device was moved in the trap as in Example 2.

The assay signal was generated and observed in a manner similar to Example 2. Differences are noted here. First, the device was inserted into the adjustable lens holder and moved into the magnetic trap. Then the suspension of Dynabead-BNP anitbody+antigen complex was pipeted into the device through the hole in the lid. The suspension was observed to flow down the lane by capillary action and reached the location of the detectable label (the “FETL spot”) in 10-20 seconds. During the flow process, the magnetic trap was located at a position approximately half way between the sample entry hole and the FETL spot. This prevented the magnetic particles from reaching the FETL spot before they were moved there by the magnets. The device was then moved through the magnetic trap according to a predetermined series of moves, similar to Example 2. These caused the magnetic particles to move through the lane at 1 mm per second. The magnetic particles were allowed to incubate with the detectable label and sample for 120 seconds. After removing the device from the magnetic trap, the device was scanned for fluorescence signal. A signal peak was observed in the location of the magnetic particles.

FIG. 13 shows a graph of the signal strength vs. BNP concentration. The magnetic particles were not casein blocked in this data set. From 2 to 4 devices were run at each BNP concentration. In the figure, the black circles indicate signal averages while the error bars indicate one standard deviation. As in the previous examples, the signal strength correlated with the concentration of BNP.

FIG. 14 shows another data set using casein blocked magnetic particles. The results are from five different lots of devices. From each device lot, 8 devices were run with BNP concentration=223 pg/mL and another 8 devices with BNP concentration<5 pg/mL. The figure shows typical variability between device lots. The following table gives the CV's (coefficient of variance) for the high calibrator (223 pg/mL), for each device lot. It also gives the minimum detectability in units of BNP pg/ml. Minimum detectability was computed as two times the standard deviation of the zero calibrator signal divided by the mean of the high calibrator signal multiplied by 223 pg/ml.

| Device Lot Number | 1 | 2 | 3 | 4 | 5 |
|---|---|---|---|---|---|
| CV | 10.6% | 14.6% | 10.6% | 5.1% | 11.7% |
| Minimum | 10.6 | 6.0 | 4.7 | 6.3 | 5.1 |
| Detectability | | | | | |
| (pg/ml) | | | | | |

### Example 5

### Detection of BNP Antigen Using Reconstituted Magnetic Beads

Preparation of Devices with Microfluidic Channel

The devices used in this example were made from parts similar to those used in the manufacture of Biosite's TRIAGE® devices with modifications as discussed below. The only modification to the base was the lane as it had a depth of 50 μm, which was deeper than a standard device. The lane was 2 mm wide in this example. The lids used were the same as described in Example 4 with the addition of a scratch in the lid<1 mm up-lane from the lid through-hole. The scratch prevented fluid from traveling up-lane.

The steps used to create the modified devices were as follows, with reference to the various reagents described in Example 1: 1. Spray the cleaned base with casein solution. 2. Spray the lid with lid spray. 3. With a Pipetman, spot approximately 0.3 μL of 68 nm diameter BNP-FETL on the upward end of the lane. The FETL spots were 3 mm long and centered approximately 24 mm from noise of the base. 5. With a Pipetman, spot approximately 0.5 μL of additional surface treatment (see below) on the lane over a length of 8 mm, centered at 52 mm from the nose of the base, and let dry. 6. Spot approximately 0.25 μL of bead suspension over the additional surface treatment and let dry. 7. Weld the lid to the base.

The additional surface treatment mentioned in the paragraph above contained the same ingredients used in the BNP-FETL, but without the FETL particles. The magnetic particle suspension was the same as described in Example 4, without the casein block.

### Detection of Fluorescence Signal

Fluorescence Signal was detected in the same manner as in Example 4 using the magnetic trap as also described in Example 4. The assay signal was measured similarly to that described in Example 4. The main difference was that the magnetic particles were not incubated with the BNP standard calibration solution outside of the device. Instead the neat calibration solution was added to the device. As the solution flowed past the location in the device in which magnetic particles had been positioned, approximately half of the beads reconstituted into the sample. The beads then began to flow down the lane; however, the magnet trap was used to prevent them from flowing down to the location in the device in which the detectable label (anti-BNP FETL) had been positioned. This gave the magnetic particles time to incubate with the sample before incubating with the FETL. After the sample finished flowing down the lane and the magnetic particles reconstituted, the magnetic particles were moved through the calibration solution down to the FETL location, where they were incubated for 120 seconds. Then the reconstituted magnetic particles were moved to a location 5 mm down-lane.

After removing the device from the magnetic trap, the device was scanned for fluorescence signal. A signal peak was observed in the location of the reconstituted magnetic particles that were positioned using the magnetic trap at the detection location.

FIG. 15 shows a graph of the signal strength vs. BNP concentration. From 4 to 8 devices were run at each BNP concentration. In the figure, the black circles indicate signal averages while the error bars indicate one standard deviation. The plot is similar to the dose response curve in Example 4, demonstrating that reconstituted magnetic particles prepositioned in the device and dried reconstitute and behave similarly to magnetic particles that were not prepositioned in the device.

While the invention has been described and exemplified in sufficient detail for those skilled in this art to make and use it, various alternatives, modifications, and improvements should be apparent without departing from the spirit and scope of the invention.

One skilled in the art readily appreciates that the present invention is well adapted to carry out the objects and obtain the ends and advantages disclosed, as well as those inherent therein. The examples provided herein are representative of suitable embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications can be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably can be practiced in the absence of any element or elements, limitation or limitations which is not specifically disclosed herein. Thus, for example, in each instance herein any of the terms “comprising”, “consisting essentially of and “consisting of may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed can be resorted to by those skilled in the art, and that such modifications and variations are considered to be within the scope of this invention as defined by the appended claims.

Other embodiments are set forth within the following claims.

1. A method for performing an assay for an analyte in a fluid sample, comprising:
(a) introducing said fluid sample into an assay device comprising:
(i) a sample addition region to receive said fluid sample,
(ii) a second device region discrete from, and in fluid communication with, said sample addition region, said second device region comprising a population of labeled conjugates corresponding to said analyte, and
(iii) an analyte detection region discrete from, and in fluid communication with, both said sample addition region and said second device region,
by applying said fluid sample to said sample addition region, wherein said assay device is configured to provide, upon application of said fluid sample to said sample addition region, fluid flow from said sample addition region to said second device region, thereby contacting said labeled conjugates in said second device region with at least a portion of said fluid sample, and fluid flow from said sample addition region to said analyte detection region;
(b) contacting said labeled conjugates with a population of magnetically responsive particles in the presence of at least a portion of said fluid sample, thereby forming a reaction mixture in said second device region, wherein said magnetically responsive particles are configured to form a complex with said labeled conjugates in an amount related to the presence or amount of said analyte in said reaction mixture;
(c) applying a magnetic field to said assay device, said magnetic field configured to induce movement of said magnetically responsive particles on a path from said second device region to said analyte detection region, wherein the direction of said movement is counter to the direction of said fluid flow from said sample addition region to said second device region for at least a portion of said path; and
(d) detecting a signal from labeled conjugates in said analyte detection region.

2. A method according to claim 1, wherein at least one of said second device region or analyte detection region is within an enclosed chamber, wherein said enclosed chamber is mesoscale in at least one dimension.

3.-4. (canceled)

5. A method according to claim 1, wherein at least one of said second device region or analyte detection region is within an enclosed chamber of said assay device comprising at least one dimension less than 100 μm.

6. A method according to claim 1, wherein said device comprises a chamber that is substantially elongated through which said magnetically responsive particles are moved.

7. A method according to claim 6, wherein said chamber comprises an aspect ratio of at least about 5.

8.-11. (canceled)

12. A method according to claim 1, wherein said assay device is configured to provide passive fluid flow from said sample addition region to one or both of said second device region and said analyte detection region, and wherein said passive fluid flow is mediated by capillary force, hydrostatic force, or by a combination of these forces.

13.-15. (canceled)

16. A method according to claim 1, wherein said assay device is configured to provide active fluid flow from said sample addition region to one or both of said second device region and said analyte detection region, and
wherein said active fluid flow is mediated by application of force generated via mechanical pumps, electroosmotic pumps, centrifugal force, increased air pressure, or by a combination of two or more such forces.

17. (canceled)

18. A method according to claim 1, wherein said assay device is configured to provide both active and passive fluid flow from said sample addition region to one or both of said second device region and said analyte detection region.

19. (canceled)

20. A method according to claim 1, wherein said population of magnetically responsive particles are spherical, and
wherein said population of magnetically responsive particles comprise a size range of between about 0.1 to 100 μm in diameter.

22.-25. (canceled)

26. A method according to claim 1, wherein said population of magnetically responsive particles is disposed within said assay device prior to application of said fluid sample.

27. (canceled)

28. A method according to claim 1, wherein said magnetically responsive particles are prepositioned on a surface of said assay device prior to applying said fluid sample, and upon application of said fluid sample to said sample addition region, fluid flowing from said sample addition region to said second device region dissolves said magnetically responsive particles into said fluid sample prior to step (b);
wherein step (b) comprises moving said magnetically responsive particles within a substantially elongated device chamber to said second device region; and
wherein step (c) comprises moving said magnetically responsive particles within said substantially elongated device chamber to said analyte detection region.

29. A device for performing an assay for an analyte in a fluid sample, comprising:
a sample addition region to receive said fluid sample;
a second device region discrete from, and in fluid communication with, said sample addition region, said second device region comprising a population of labeled conjugates corresponding to said analyte; and
an analyte detection region discrete from, and in fluid communication with, both said sample addition region and said second device region; and
magnetically responsive particles disposed within said device, said particles comprising receptors immobilized thereon so as to be configured to form a complex with said labeled conjugates during performance of said assay;
wherein said analyte detection region is positioned in said device relative to said sample addition region and said second device region such that a first path for movement of material from said second device region to said analyte detection region is counter in direction to a second path for movement for material from said sample addition region to said second device region for at least a portion of said first path, and
wherein said sample addition region further comprises a filter element or a vent, and
wherein said sample addition region comprises a volume capacity that is at least one times the volume capacity of the second device region or the analyte detection region.

30.-38. (canceled)

39. An assay system for performing an assay for an analyte in a fluid sample, comprising:
a device according to claim 29, wherein said labeled conjugates comprise a label moiety that generates a detectable optical signal upon illumination with electromagnetic energy having a wavelength that is absorbed by said label moiety; and
an assay instrument comprising:
a receptacle for receiving said device,
a magnetic field source that generates a magnetic field having an intensity sufficient to induce movement of said magnetically responsive particles on a path from said second device region to said analyte detection region during performance of said assay,
a source of electromagnetic energy positioned to illuminate said analyte detection region during performance of said assay to generate a detectable optical signal from said labeled conjugates in said analyte detection region;
a detector positioned to receive said detectable optical signal and generate an electronic signal in response thereto.

40. The assay system of claim 39, wherein the magnetic field source comprises a permanent magnet, a ferrometallic alloy, a ceramic ferrite, or a rare earth alloy; and wherein the intensity of said magnetic field source is controlled by a relative positioning of the magnetic field source with respect to the device, or is controlled by a movable shield, or is controlled electronically.

41.-46. (canceled)
